(19) United States
(12) Patent Application Publication
(10) Pub. No.: US 2016/0158174 A1
(43) Pub. Date: Jun. 9, 2016

(54) Enteric-Coated Functional Food Ingredients And Methods For Making The Enteric-Coated Functional Food Ingredients

(71) Applicant: Intercontinental Great Brands LLC, East Hanover, NJ (US)

(72) Inventors: David K. Hayashi, East Hanover, NJ (US); Ahmad Akashe, East Hanover, NJ (US); Anilkumar Gaonkar, East Hanover, NJ (US)

(21) Appl. No.: 14/564,726

(22) Filed: Dec. 9, 2014

(51) Int. Cl. A61K 31/19; A23L 1/30; A23L 1/00; A61K 9/48

(52) U.S. Cl. CPC A61K 31/19; A61K 9/4866; A23L 1/30; A23L 1/0029; A23V 2002/00

(57) ABSTRACT

Functional food ingredients for delivery to the gastrointestinal tract are delivered. Food products, nutraceuticals, and pharmaceuticals comprising the functional food ingredients, as well as methods for making the functional food ingredients, are also provided. The functional food ingredients may positively influence glucose metabolism and weight management. Generally, the ingredients include metabolites physically entrapped in a fermentation precursor, which is then encapsulated in an enteric coating for release in the large intestine of a human subject. In one approach, the ingredients include a polysaccharide matrix, short chain fatty acids physically entrapped in the polysaccharide matrix, and an enteric coating that encapsulates the combination of short chain fatty acids and polysaccharide matrix.

### FIELD

The disclosure relates to enteric-coated functional food ingredients and particularly compositions comprising metabolites entrapped in a fermentation precursor matrix that is enteric coated for targeted release in the large intestine after consumption.

### BACKGROUND

In recent years, there's been an increase in consumer interest in products that promote gut health. Trends indicate that consumer interest in probiotic and/or prebiotic products will continue to grow as consumers become better educated on the health benefits provided by gut microbiota. These products may include one or both of prebiotics and probiotics. Generally, probiotics include live bacteria, and prebiotics include non-digestible ingredients, such as dietary fibers, that stimulate the growth of gut microbiota. Probiotics are often found in fermented foods, drinkable and spoonable yogurt and beverage products, as well as in other foods like sauerkraut and some soft cheeses, while prebiotics can be found in plant-based foods, such as whole grains, bananas, artichokes, garlic, and legumes. Probiotics are also readily available in the form of dietary supplements. Probiotic and prebiotic products are available in a variety of formats, including both consumer and clinical applications, such as oral, enteral, and rectal formulations.

Probiotic and/or prebiotic products are reported to provide a number of health benefits, including improved digestion, nutrient absorption, and ability to fend off infection by harmful microorganisms. Gut health is an active area of scientific study. Probiotic and prebiotics have been investigated for treatment of other ailments, including irritable bowel syndrome, ulcerative colitis, Crohn's disease, and food allergies.

There has also been increased investigation into the potential effects of gut microbiota on metabolism and immunity, as well as obesity, inflammation, cardiovascular disease and diabetes. One area of investigation is the production of short chain fatty acids (SCFA) by gut microbiota as byproducts of the breakdown of dietary fiber to prevent the onset of type two diabetes. It is believed that recognition of SCFAs by receptors on intestinal epithelial cells turn on systemic biochemical signals to positively regulate glucose metabolism and direct the expenditure of host energy metabolism away from fat storage. SFCAs are also believed to act as an antimicrobial agent toward select fungi and bacteria at low pH when they are in their dissociated form to advantageously modulate the gut microbiota in favor of beneficial microbes.

Specifically it has been reported that SFCAs stimulate glucagon-like peptide 1 (GLP-1) secretion from primary colonic cultures. G. Tolhurst et al., “Short-Chain Fatty Acids Stimulate Glucagon-Like Petptide-1 Secretion via the G-Protein-Coupled Receptor FFAR2,” Diabetes, 61: 364-371 (2012). GLP-1 mimetics have been reported to be associated with improved blood glucose control.

Current western diets low in dietary fiber are generally thought of as not being capable of providing the necessary precursors to support beneficial gut microbes and their production of SCFAs. Further, SCFAs on their own have a distinct taste and flavor profile which would not be acceptable to many consumers.

Some have investigated ways to deliver SCFAs to the gut. For example, U.S. Patent Application Publication No. 2006/0280776 describes diet foods having the effect of reducing body weight and preventing and/or improving obesity and atherosclerotic or metabolic disorders. The diet food includes an ω-3 polyunsaturated fatty acid or an ω-6 polyunsaturated fatty acid, and at least one of L-arginine, L-ornithine, L-arginine precursor, and L-ornithine precursor. In another approach, the diet food may include diacylglycerol, a middle or short chain fatty acid, phytosterol, and at least one of L-arginine, L-ornithine, L-arginine precursor, and L-ornithine precursor. The diet food may also include soluble fibers such as pectin, guar gum, and locust bean gum.

U.S. Pat. No. 6,994,869 describes a nasogastric formulation comprising an amino acid source, a carbohydrate source, a lipid source, and a fatty acid delivery agent for delivery of fatty acids to the large bowel. The fatty acids in the fatty acid delivery agent are covalently bonded to a carrier by a bond that is hydrolysable in the colon to release free fatty acids. The bond is described as an amide or ester bond. The carrier is described as including natural dietary fiber or non-digestible oligosaccharides, such as inulin, chitin, beta-glucans, mucilages, agar, carrageenans, and gums including guar, arabic, xanthan, tragacanth, locust bean, and psyllium.

The efficacy of these prior products and methods is at least partially constrained by the ability of the fatty acids to arrive in the large intestine. It is believed that the covalent bonds of the prior products such as those described in U.S. Pat. No. 6,994,869, will begin to hydrolyze when going through the stomach, thereby releasing the fatty acids which will then largely be absorbed by the body at the point of hydrolysis. To improve efficacy, it is presently believed that these covalent bonds would need to arrive intact in the large intestine after another approximately six hours of transit to provide the desired absorption by the large intestine. Therefore, the prior attempts to deliver SFCAs to the gut will generally provide limited bioavailability and efficacy due to hydrolysis in the stomach and small intestine.

### SUMMARY

Disclosed herein are enteric-coated compositions effective to deliver metabolites to the large intestine of a human subject. In one aspect, the enteric-coated composition may be considered a functional food ingredient. In some approaches, the enteric-coated compositions are effective to deliver metabolites and fermentation precursors to the gastrointestinal tract in order to positively influence glucose metabolism and weight management. Generally, the enteric-coated compositions include metabolites physically entrapped in a fermentation precursor, which is then encapsulated in an enteric coating for release in the large intestine of a human subject. In one approach, the composition includes a polysaccharide matrix, short chain fatty acids physically entrapped in the polysaccharide matrix, and an enteric coating that encapsulates the combination of short chain fatty acids and polysaccharide matrix.

In one approach, a functional food ingredient comprises a fermentation precursor matrix comprising a metabolite entrapped in the fermentation precursor matrix and an enteric coating that encapsulates the fermentation precursor matrix with the entrapped metabolite.

In another approach, the enteric-coated functional food ingredients include about 1 to about 50 percent metabolite, in another aspect about 5 to about 40 percent metabolite, in yet another aspect about 10 to about 30 percent metabolite; about 5 to about 90 percent fermentation precursor, in another aspect about 15 to about 70 percent fermentation precursor, in yet another aspect about 25 to about 60 percent fermentation precursor; and about 1 to about 70 percent enteric coating, in another aspect about 5 to about 60 percent enteric coating, in yet another aspect about 10 to about 50 percent enteric coating, with all percentages based on the total weight of the enteric-coated functional food ingredient.

In another aspect, a method of suppressing appetite in a human subject, such as by regulating glucose metabolism of a human subject by activating at least one free fatty acid receptor selected from the group consisting of FFAR2 and FFAR3 is provided. The method comprising administering to the human subject a composition comprising a fermentation precursor matrix comprising a metabolite entrapped in the fermentation precursor matrix; and an enteric coating that encapsulates the fermentation precursor matrix with the entrapped metabolite.

In one aspect, the fermentation precursor matrix comprises at least one polysaccharide selected from the group consisting of pectin, alginate, xylan, guar gum, or a combination thereof. In another aspect, the metabolite is selected from the group consisting of propionic acid or salt thereof, butyric acid or salt thereof, acetic acid or salt thereof, lactic acid or salt thereof, succinic acid or salt thereof, or a combination thereof. In one approach, the metabolite comprises sodium propionate and the fermentation precursor matrix comprises pectin, such as low methoxyl pectin or high methoxyl pectin. In one aspect, the low methoxyl pectin matrix may be crosslinked with a metal divalent or trivalent cation.

The fermentation precursor matrix with entrapped metabolite is coated with an enteric coating. The enteric coating as used to encapsulate the combination of metabolites and fermentation precursors described herein may be formulated such that the coating does not dissolve, or at most minimally dissolves, in the stomach of a human subject following oral administration. Generally, the enteric coating may include any food grade enteric polymer or a combination or two or more food grade enteric polymers. For example, suitable enteric coating materials include shellac, zein, ethyl cellulose, or combinations thereof. As discussed below, the relative amounts of the enteric coating materials can be selected to achieve the desired rate of degradation after ingestion. In one particular aspect, the enteric coating includes an inner layer comprising ethyl cellulose, a middle layer comprising zein, and an outer layer comprising shellac.

The enteric coating may be formulated to provide minimal release of the metabolites as the enteric coated composition passes through the stomach and to at least partially degrade as the composition passes through the small intestine. In one approach, the enteric coating is formulated such that less than about 25%, in another aspect less than about 20%, in another aspect less than about 15%, in another aspect less than about 10%, and in yet another aspect less than about 5% of the metabolite in the composition is released in the stomach after consumption. It is generally desired that a substantial portion of the metabolites are released in the large intestine after degradation of the enteric coating to expose the fermentation precursor matrix with the metabolites entrapped therein. In one approach, the enteric coating is formulated such that at least 10 percent, in another aspect at least about 20 percent, in another aspect at least about 30 percent, in another aspect at least about 40 percent, in another aspect at least about 50 percent, and in yet another aspect at least about 60 percent of the metabolites in the composition are released in the large intestine.

The enteric-coated fermentation precursor matrix with entrapped metabolite can be provided in a food product, pharmaceutical, or nutraceutical product. In one aspect, the food product is a chewing gum, biscuit, cookie, powder beverage, chocolate, or confection.

In another aspect, a method of making an enteric-coated functional food ingredient is provided. The method includes heating an aqueous liquid to a temperature of about 50° C. to about 80° C.; adding a fermentation precursor to the heated aqueous liquid to form a first mixture; adding a metabolite to the first mixture to form a second mixture; drying the second mixture to form a powder; milling the dried powder to provide particles; and coating the particles with an enteric coating. In one approach, the fermentation precursor matrix comprises at least one polysaccharide selected from the group consisting of pectin, alginate, xylan, guar gum, or a combination thereof. In another approach, the metabolite is selected from the group consisting of propionic acid or salt thereof, butyric acid or salt thereof, acetic acid or salt thereof, lactic acid or salt thereof, succinic acid or salt thereof, or a combination thereof. The method may further comprise adding a binder solution including a metal divalent or trivalent cation after the drying step. The method may also further comprise adjusting a pH of the first mixture to about 6.0 to about 7.5 prior to adding the metabolite to the first mixture.

The enteric-coated functional food ingredient can be provided in the form of particles of desired size. For example, particles having a median diameter of about 50 microns to about 3 mm, in another aspect about 100 microns to about 3 mm may be obtained. If microparticles are desired, the dried powder can be milled to a median diameter of about 50 to about 500 microns, in another aspect about 100 to about 500 microns, and in another aspect about 200 to about 500 microns.

### BRIEF DESCRIPTION OF THE DRAWINGS

FIG. 1 provides a schematic of the configuration of an exemplary enteric-coated composition as it passes from the stomach to large intestine.

FIG. 2 includes a flow diagram of an exemplary method of making a composition including a metabolite physically entrapped in a fermentation precursor matrix.

FIG. 3 includes a flow diagram of an exemplary method of making an enteric-coated composition including short chain fatty acids physically entrapped in a pectin matrix.

FIG. 4 includes an exemplary modified Simulator of Human Intestinal Microbial Ecosystem (“SHIME”®) setup.

FIG. 5 includes a scatter graph showing the concentration of propionate measured during an in vitro digestion evaluation in a simulated stomach and small intestine using a SHIME setup, with treatment with a control sample (Ctrl), low methoxyl (LM) pectin sample, or high methoxyl (HM) pectin sample.

FIGS. 6A-6F include scatter graphs showing the concentrations of acetate, propionate, and butyrate in a simulated proximal and distal colon over two weeks as measured during an in vitro digestion evaluation in a SHIME setup: FIG. 6A shows the graph for the proximal colon treated with the control; FIG. 6B shows the graph for the distal colon treated with the control; FIG. 6C shows the graph for the proximal colon treated with the low methoxyl pectin (“LM”) sample; FIG. 6D shows the graph for the distal colon treated with the LM sample; FIG. 6E shows the graph for the proximal colon treated with the high methoxyl pectin (“HM”) sample; and FIG. 6F shows the graph for the distal colon treated with the HM sample.

FIG. 7 includes a bar graph illustrating the concentration of propionate in the simulated proximal colon as measured during an in vitro digestion evaluation in a SHIME setup after treatment with a control sample, low methoxyl pectin sample, and high methoxyl pectin sample.

FIG. 8 includes a bar graph illustrating the concentration of propionate in the simulated distal colon as measured during an in vitro digestion evaluation in a SHIME setup after treatment with a control sample, low methoxyl pectin (“Low”) sample, and high methoxyl pectin (“High”) sample.

FIGS. 9A-9F include bar graphs showing the concentrations of butyrate, propionate, and acetate in a simulated proximal and distal colon over two weeks as measured during an in vitro digestion evaluation in a SHIME setup: FIG. 9A shows the graph for the proximal colon treated with the control; FIG. 9B shows the graph for the distal colon treated with the control; FIG. 9C shows the graph for the proximal colon treated with the low methoxyl pectin (“LM”) sample; FIG. 9D shows the graph for the distal colon treated with the LM sample; FIG. 9E shows the graph for the proximal colon treated with the high methoxyl pectin (“HM”) sample; and FIG. 9F shows the graph for the distal colon treated with the HM sample.

FIG. 10A-10C include bar graphs illustrating the concentration of total lactic acid in the simulated proximal colon and the distal colon as measured during an in vitro digestion evaluation in a SHIME setup: FIG. 10A shows the lactic acid concentrations for the control sample; FIG. 10B shows the lactic acid concentrations for the low methoxyl pectin (“LM”) sample; and FIG. 10C shows the lactic acid concentrations for the high methoxyl pectin (“HM”) sample.

FIG. 11A-11C include bar graphs illustrating the concentrations of ammonium in the simulated proximal colon and the distal colon as measured during an in vitro digestion evaluation in a SHIME setup for the control (FIG. 11A), after treatment with a low methoxyl pectin sample (FIG. 11B), and after treatment with a high methoxyl pectin sample (FIG. 11C).

FIG. 12 includes a bar graph illustrating the concentrations of total bacteria, Bacteroidetes bacteria, and Firmicutes bacteria in the simulated proximal colon before and after treatment with a control sample.

FIG. 13 includes a bar graph illustrating the concentrations of total bacteria, Bacteroidetes bacteria, and Firmicutes bacteria in the simulated distal colon before and after treatment with a control sample.

FIG. 14 includes a bar graph illustrating the concentrations of total bacteria, Bacteroidetes bacteria, and Firmicutes bacteria in the simulated proximal colon before and after treatment with a low methoxyl pectin sample.

FIG. 15 includes a bar graph illustrating the concentrations of total bacteria, Bacteroidetes bacteria, and Firmicutes bacteria in the simulated distal colon before and after treatment with a low methoxyl pectin sample.

FIG. 16 includes a bar graph illustrating the concentrations of total bacteria, Bacteroidetes bacteria, and Firmicutes bacteria in the simulated proximal colon before and after treatment with a high methoxyl pectin sample.

FIG. 17 includes a bar graph illustrating the concentrations of total bacteria, Bacteroidetes bacteria, and Firmicutes bacteria in the simulated distal colon before and after treatment with a high methoxyl pectin sample.

FIGS. 18A-18C show bar graphs illustrating the concentrations of Lactobacilli in the simulated proximal and distal colon before and after treatment with a control sample (FIG. 18A), low methoxyl pectin sample (FIG. 18B), and high methoxyl pectin sample (FIG. 18C).

FIG. 19A-19C show bar graphs illustrating the concentrations of Bifidobacteria in the simulated proximal and distal colon before and after treatment with a control sample (FIG. 19A), low methoxyl pectin sample (FIG. 19B), and high methoxyl pectin sample (FIG. 19C).

### DETAILED DESCRIPTION

Provided herein are functional food ingredients for delivery to the gastrointestinal tract that may positively influence glucose metabolism and weight management. Generally, the ingredients include metabolites physically entrapped in a fermentation precursor, which is then encapsulated in an enteric coating for release in the large intestine of a human subject. In one approach, the composition includes a polysaccharide matrix, short chain fatty acids physically entrapped in the polysaccharide matrix, and an enteric coating that encapsulates the combination of short chain fatty acids and polysaccharide matrix.

In one approach, the enteric-coated functional food ingredients include about 1 to about 50 percent metabolite, in another aspect about 5 to about 40 percent metabolite, in yet another aspect about 10 to about 30 percent metabolite; about 5 to about 90 percent fermentation precursor, in another aspect about 15 to about 70 percent fermentation precursor, in yet another aspect about 25 to about 60 percent fermentation precursor; and about 1 to about 70 percent enteric coating, in another aspect about 5 to about 60 percent enteric coating, in yet another aspect about 10 to about 50 percent enteric coating, with all percentages based on the total weight of the enteric-coated functional food ingredient.

As used herein, the term “gastrointestinal tract” includes the stomach, small intestine, and large intestine (which includes the proximal colon and the distal colon). As used herein, the term “intestines” includes the small intestine and large intestine (which includes the proximal colon and the distal colon).

As used herein, the term “metabolite” includes short chain fatty acids and their derivatives and salts (e.g., propionic acid, butyric acid, acetic acid, sodium propionate, calcium propionate, or the like), as well as lactic and succinic acid and salts thereof, as well as any other products or byproducts of gut microbial bioconversion processes. As used herein, the term “short chain fatty acid” includes fatty acids with aliphatic tails of fewer than six carbons, including, but not limited to acetic acid, propionic acid, and butyric acid, and combinations thereof, and their salts, including, but not limited to propionate, butyrate, and acetate, and combinations thereof.

In the compositions described herein, one or more metabolites may be embedded into a fermentation precursor matrix. As used herein, the term “fermentation precursor” includes components which provide a substrate for microbial fermentation in the intestines by being a source of nutrients for gut microbiota. Preferred fermentation precursors include those that can form a structural matrix capable of physically entrapping a metabolite therein. In one particular aspect, the metabolite may be entrapped and dispersed in the matrix without a covalent bond being formed between the metabolite and the fermentation precursor.

For example, polysaccharides may be used as the fermentation precursor. Generally, polysaccharides are polymeric carbohydrate molecules that include long chains of monosaccharide units bound together by glycosidic linkages. Polysaccharides may have a linear or branched structure. Exemplary polysaccharides include storage polysaccharides such as starch and glycogen and structural polysaccharides such as cellulose and chitin. In one form, the composition as described herein includes one or more structural polysaccharides including, but not limited to pectin, alginate, xylan, and guar gum. At least in some approaches, it will be appreciated that a variety of matrix ingredients other than fermentation precursors may be used so long as the matrix ingredient is able to entrap the metabolite and release the metabolite in the large intestine. In preferred approaches, one or more polysaccharides that are fermentable by gut microbiota are used to provide the structural matrix for the incorporation of the short chain fatty acids because the consumption (i.e., fermentation) of the matrix ingredient by the intestinal bacteria provides for a desired controlled release of the metabolites (e.g., short chain fatty acids).

In one approach, when the fermentation precursor is a polysaccharide, fermentation of the polysaccharide by the gut microbiota in the large intestine may result in the production of short chain fatty acids. As such, the compositions including short chain fatty acids embedded in the polysaccharides as described herein, when delivered to the intestines of the human subject following oral administration, may advantageously provide a source of short chain fatty acids not only directly but also indirectly via the fermentation of the polysaccharides in the large intestine. In addition, the short chain fatty acids, when delivered to the large intestine of a human subject, may act as antimicrobial agents toward certain sensitive strains of microbes and may thus advantageously modulate the make-up of the microbiota of the gut.

Pectin is a structural heteropolysaccharide contained in the primary cell walls of many terrestrial plants. In the compositions described herein, high methoxyl pectin and/or low methoxyl pectin may be used. As used herein, the term “low methoxyl pectin” refers to pectins where a relatively low portion (i.e., less than 50%) of the carboxyl groups of all the galacturonic acid present in the pectin is esterified as methyl esters. As used herein, the term “high methoxyl pectin” refers to pectins where a relatively high portion (i.e., 50% or more) of the carboxyl groups of all the galacturonic acid present in the pectin is esterified as methyl esters.

In one approach, a composition for delivery to the intestines of a human subject is provided. In one aspect, the composition may include a fermentation precursor, a metabolite entrapped in the fermentation precursor, and an enteric coating that encapsulates the combination of the fermentation precursor and metabolite. In one particular aspect, the composition may include a polysaccharide, a short chain fatty acid or a salt of a short chain fatty acid entrapped in the polysaccharide, and an enteric coating that encapsulates the combination of the polysaccharide and the short chain fatty acid.

The compositions described herein may be orally administered in the form of a pharmaceutical, nutraceutical, or dietary supplement, such as in the form of a pill, tablet, powder, capsule, liquid mixture or solution, or may be added to food products such as biscuits, snacks, crackers, chocolates, confectioneries, cookies, chewing gum, powdered beverage mixes, dry seasoning blends, or other food or beverage product. In some approaches, these food products containing the compositions provided herein may be considered functional foods. Generally, the term “functional foods” as used herein refers to food or beverage products that provide a potentially beneficial effect on health beyond basic nutrition, such as to provide beneficial effect for disease or promote improved health or body function. The entrapment of the metabolites in the fermentation precursor as described herein may also advantageously mask inherent negative organoleptic characteristics of the metabolites, particularly short chain fatty acids, and may allow the compositions to be incorporated into a variety of food products without detrimentally affecting the flavor or organoleptic properties of the products.

As noted above, the fermentation precursor matrix may provide a substrate for fermentation by being a source of nutrients for the microbiota of the large intestine. In one approach, the compositions containing the metabolites entrapped in the fermentation precursor matrix are formulated so that the metabolites are not released, or are at least minimally released, from the fermentation precursor matrix into the small intestine so that a substantial portion of the metabolites are delivered to the large intestine, particularly, to the proximal colon and distal colon as described in more detail below.

The short chain fatty acids as used in the compositions described herein, when delivered to the large intestine of the human subject following oral administration, may advantageously activate receptors on intestinal epithelial cells. It is believed that the enteric-coated compositions described herein may be used to depress appetite and, therefore, may be used to promote weight loss. For example, the short chain fatty acids may activate free fatty acid receptors such as FFAR2 and FFAR3 in the large intestine, in particular, in the colon. The activation of the FFAR2 and/or FFAR3 receptors in the colon may trigger secretion of at least one of glucagon-like peptide (GLP)-1 and peptide YY (PYY) from the intestinal epithelial cells of the human subject.

GLP-1 is known to induce glucose-dependent stimulation of insulin secretion from the pancreas while suppressing glucagon secretion from the pancreas and has been shown to stimulate the feeling of satiety in human subjects. PYY is known to inhibit gastric motility and increases water and electrolyte absorption in the colon and has been shown to reduce appetite. As such, the delivery of the short chain fatty acids to the large intestine as described herein may initiate biochemical signalling pathways which positively regulate glucose metabolism and direct the expenditure of host energy metabolism away from fat storage.

The enteric coating as used to encapsulate the combination of metabolites and fermentation precursors described herein may be formulated such that the coating does not dissolve, or at most minimally dissolves, in the stomach of a human subject following oral administration. Generally, the enteric coating may include any food grade enteric polymer or a combination or two or more food grade enteric polymers. For example, suitable enteric coating materials include shellac, zein, ethyl cellulose, or combinations thereof. As discussed below, the relative amounts of the enteric coating materials can be selected to achieve the desired rate of degradation after ingestion.

Shellac and zein undergo pH-dependent solubilization and are expected to begin to dissolve and solubilize at least partially as compositions coated with shellac and/or zein pass through the small intestine. In one approach, the shellac can be provided as an alkaline (pH>7) aqueous solution, such as a water-based solution having a solid content of about 25 percent by weight or it can be prepared from refined, bleached and dewaxed shellac powder. Degradation of ethyl cellulose differs in that it is not pH-dependent. Instead, ethyl cellulose is not water soluble and breaks down by erosion and diffusion in a time dependent process. Therefore, combinations of the enteric coating materials can be selected so as to provide the desired degradation as the product passes through the stomach and small intestine. Similarly, the amounts of each material used (e.g., thickness of the coatings) can also impact the degradation properties of the compositions.

The enteric coating may be formulated to provide minimal release of the metabolites as the enteric coated composition passes through the stomach and to at least partially degrade as the composition passes through the small intestine. In one approach, the enteric coating is formulated such that less than about 25%, in another aspect less than about 20%, in another aspect less than about 15%, in another aspect less than about 10%, and in yet another aspect less than about 5% of the metabolite in the composition is released in the stomach after consumption. It is generally desired that a substantial portion of the metabolites are released in the large intestine after degradation of the enteric coating to expose the fermentation precursor matrix with the metabolites entrapped therein. In one approach, the enteric coating is formulated such that at least 10 percent, in another aspect at least about 20 percent, in another aspect at least about 30 percent, in another aspect at least about 40 percent, in another aspect at least about 50 percent, and in yet another aspect at least about 60 percent of the metabolites in the composition are released in the large intestine. The amount of metabolite released in the stomach, small intestine, and/or large intestine can be estimated as described below in Example 1.

In one particular approach, the enteric coating includes an inner coat or layer formed of ethyl cellulose, a middle coat or layer formed of zein, and an outer coat or layer made of shellac. For example, the inner coat or layer may include from about 1% to about 50% ethyl cellulose, in another aspect about 1 to about 20% ethyl cellulose, and in another aspect about 12% to about 17% ethyl cellulose. The middle coat or layer may include from about 1% to about 50% zein, in another aspect about 1% to about 20% zein, in another aspect about 5 to about 15% zein, and in yet another aspect about 8% to about 12% zein. The outer coat or layer may include from about 1% to about 50% shellac, in another aspect about 1% to about 15% shellac, and in another aspect about 10% to about 15% shellac. The percentages listed for the ethyl cellulose, shellac, and zein are based on the total weight of the composition (i.e., all enteric coating materials plus the metabolites and fermentation precursor).

It is to be appreciated that, at least in some approaches, the materials used for the layers may be interchangeable, particularly the shellac and zein layers. It will also be appreciated that the percentages of the ethyl cellulose, zein, and shellac are being shown by way of example only, and that the enteric coating may include any of ethyl cellulose, zein, and shellac in amounts outside of the exemplary ranges provided herein so long as the enteric coating is effective to deliver the metabolites entrapped in the fermentation precursor matrix substantially intact to the large intestine.

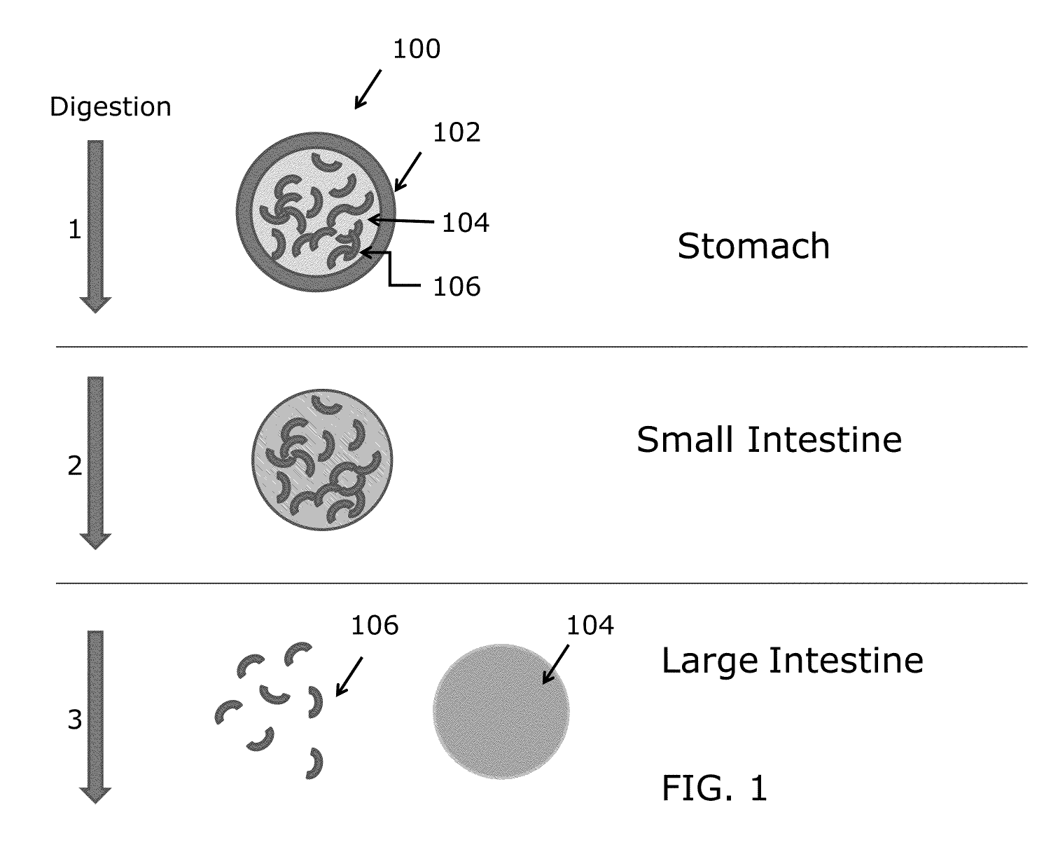

FIG. 1 includes a schematic of the configuration of an exemplary enteric-coated composition as it passes from the stomach to large intestine in accordance with at least some embodiments described herein. As shown therein, enteric coated composition 100 includes enteric coating 102, fermentation precursor matrix 104, and metabolites 106. The enteric coating 102 may include one more enteric coating materials and/or layers of enteric coating materials. The metabolites 106 are entrapped in the fermentation precursor matrix 104. Although not shown in FIG. 1, the enteric coating could be at least partially intact in the small intestine and large intestine prior to completion of the breakdown of the enteric coating in the large intestine.

In one approach, the fermentation precursor matrix 104 is a pectin-based polysaccharide matrix, such as low or high methoxyl pectin, the metabolites 106 include sodium propionate, and the enteric coating 102 includes a combination of layers of ethyl cellulose, zein, and shellac. This is an exemplary formulation for the composition, but other fermentation precursor matrix ingredients, metabolites, and enteric coating materials may be used, if desired.

In one aspect, the compositions may be provided in the form of particles, and in another aspect in the form of microparticles. As used herein, the “particles” may have a median diameter of about 50 microns to about 3 mm, in another aspect about 100 microns to about 3 mm, and the term “particles” specifically includes microparticles. The term “microparticles” refers to particles of a narrower size range. In one aspect, the term “microparticles” refers to particles having a median diameter of about 50 to about 500 microns, in another aspect about 100 to about 500 microns, and in another aspect about 200 to about 500 microns. It is not presently believed that the size of the particles is particularly limited, except perhaps for requirements of certain machinery used (such as fluid bed processing), and in at least some approaches, smaller particles may be desired so as to avoid adding undesired texture when the particles are added to food or beverage products.

As such, the embedding of the short chain fatty acids in the polysaccharide matrix advantageously protects the short chain fatty acids from being exposed to hydrolysis and/or dissolution in the small intestine and enables the short chain free fatty acids to be effectively delivered substantially intact to the large intestine, where the short chain fatty acids may activate receptors that trigger secretion of hormones, affect microbial populations via antimicrobial effects, and provide nutrients for intestinal epithelial cells, as discussed above.

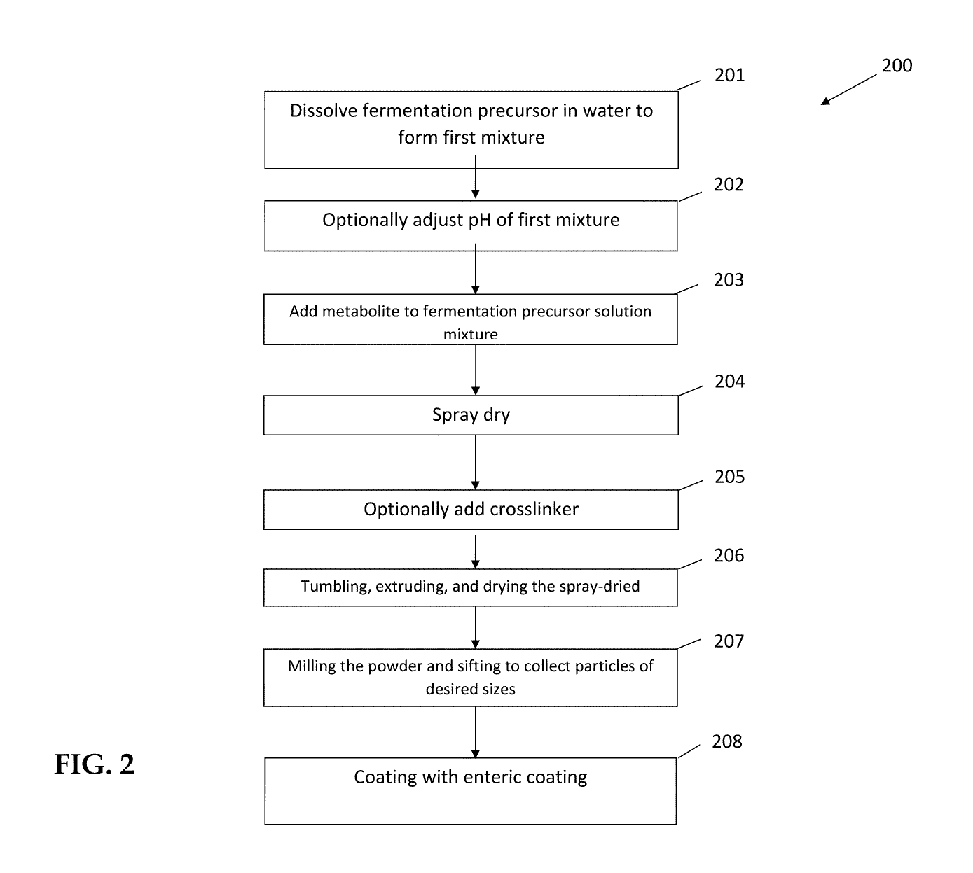

By one exemplary approach and as shown in FIG. 2, a method 200 is provided for making an enteric-coated composition including a metabolite entrapped in a fermentation precursor matrix. Generally, step 201 includes dissolving a fermentation precursor in an aqueous liquid to form a first mixture. In one approach, the fermentation precursor is soluble upon being dispersed in water at room temperature. If needed, the aqueous liquid may be pre-heated or heated after addition of the fermentation precursor to facilitate dissolution of the fermentation precursor.

In step 202, the pH of the first mixture optionally may be adjusted, as needed, depending on the fermentation precursor used. For example, for low or high methoxyl pectin, the pH of the first mixture may be adjusted to a pH of about 6 to about 7.5. Adjustment of the pH of the pectin may facilitate trapping of greater quantities of propionate or other metabolite in the pectin matrix. Pectin solutions are generally highly acidic (e.g., may have a pH between 3 and 4). If certain metabolites, such as sodium propionate, were added to an acidic pectin solution, a significant proportion of the salt will convert to more volatile propionic acid. By bringing the pH of a pectin solution to about 6.0 to about 7.5 prior to addition of the metabolite, the salt remains in a more stable form and may result in the entrapment of a greater quantity of metabolite in the pectin matrix. Appropriate pH adjustments for other polysaccharide fermentation precursors can be readily determined in the art as needed.

In step 203, the metabolite is added to the first mixture to form a second mixture. In one form, the metabolite may be lactic acid, succinic acid, a short chain fatty acid such as propionic acid, butyric acid, or acetic acid, or salt thereof. In another form, the short chain fatty acid may be a monovalent cation-based salt of the described short chain fatty acid. For example, the monovalent cation may be sodium, potassium, ammonium (such as in ammonium hydroxide), or the like. Generally, when the fermentation precursor is pectin or another crosslinkable polymer, divalent cation-based salts are less desirable than monovalent cation-based salts. Divalent cation-based salts, such as calcium salts, may result in pectin crosslinking to form a thick gel, which can result in a lesser quantity of metabolite being entrapped in the fermentation precursor matrix, as well as detrimentally affect the ease of conducting certain processing steps, such as atomization. However, divalent cation-based salts may be used in certain circumstances when processing conditions are controlled such that the desired quantity of metabolite may be entrapped in the fermentation precursor matrix.

At least in some approaches, use of metabolite salts may be more desirable than use of metabolite acids because acids can be more challenging than their respective salts to entrap in a polysaccharide matrix in desired quantities. Without wishing to be limited by theory, metabolites in acid form may be more volatile than the metabolite salts and large amounts of metabolite acids may be lost (i.e., less metabolite acids may be entrapped in the fermentation precursor matrix) as compared to the amounts of metabolite salts that may be entrapped in the fermentation precursor matrix.

In step 204, the second mixture is dried, for example, by spray-drying, freeze-drying, or the like to form a powder. For example, the second mixture can be spray-dried using a Buchii mini spray dryer model B290 at an inlet temperature from about 160° C. to about 180° C. and an outlet temperature from about 80° C. to about 90° C. Notably, freeze-drying may result in a fermentation precursor matrix with a porous structure, which may create weak points that lead to faster than desired breakdown during passage of the composition through the small intestine. It will be appreciated that when freeze-drying is used, the porosity of the composition is taken into account when formulating the desired release profile, or that freeze-drying conditions may be adjusted so as to minimize the porosity of the fermentation precursor matrix.

In optional step 205, a binder solution may be sprayed onto the powder. For example, the binder solution may be a crosslinking solution. A binder solution may comprise about 1 to about 20 percent maltodextrin (e.g., a maltodextrin having a dextrose equivalent (DE) of 10) in one aspect and about 5 to about 15 percent maltodextrin in another aspect, and about 0.2 to about 3 percent calcium chloride, but it will be appreciated that other suitable binder solutions including one or more maltodextrins, starches, carbohydrates, or proteins with a divalent or trivalent metal ion may also be used. At least in some approaches, the binder solution may assist in agglomeration of the fermentation precursor matrix, particularly when the fermentation precursor includes pectin. For example, agglomerating fine particles into larger clusters of particles may facilitate downstream processing, such as coating processes.

In some approaches, as the binder solution is being applied to the powder, the powder may be tumbled using, for example, a Hobart mixer and extruded, for example, using an LCI extruder. A further drying step 206 using, for example, a vacuum dryer, may be performed after the tumbling and extruding.

The dried powder obtained from either step 204 or 206 may then be milled in step 207 to provide particles of a desired size. For example, particles having a median diameter of about 50 microns to about 3 mm, in another aspect about 100 microns to about 3 mm may be obtained. If microparticles are desired, the dried powder can be milled to a median diameter of about 50 to about 500 microns, in another aspect about 100 to about 500 microns, and in another aspect about 200 to about 500 microns. As noted above, the size of the particles is generally not particularly limited but the size may be selected based on processing conditions or intended use of the composition. For example, very fine powders (e.g., smaller than about 50 microns) can be difficult to coat using fluid bed processing. Further, large particles may create undesirable texture to food or beverage products into which they are incorporated. The particle sizes may be measured using a variety of standard approaches, including using sieves.

The milled particles are then coated in step 208 with one or more coats of enteric coating materials. For example, a bench Mini Glatt fluid bed coater with a bottom spray Wurster process may be used. In one approach, the product temperature during coating may be about 30° C. to about 45° C. and the coating spray rate may be from about 1 g/min to about 2 g/m. Other spraying parameters

Advantages and embodiments of the enteric-coated compositions including metabolites entrapped in a fermentation precursor matrix as described herein are further illustrated by the following examples; however, the particular conditions, processing schemes, materials, and amounts thereof recited in these examples, as well as other conditions and details, should not be construed to unduly limit the compositions and methods described herein. All percentages in this application are by weight unless otherwise indicated.

### EXAMPLES

The following Examples illustrate exemplary methods of preparing an enteric-coated composition including a short chain fatty acid such as propionate embedded in the polysaccharide matrix provided by pectin. The Examples illustrate the efficacy of delivering metabolites, as well as the fermentation precursors, to the proximal and distal colon.

### Example 1

### Use of Low Methoxyl Pectin to Entrap Propionate for Targeted Delivery to the Large Intestine

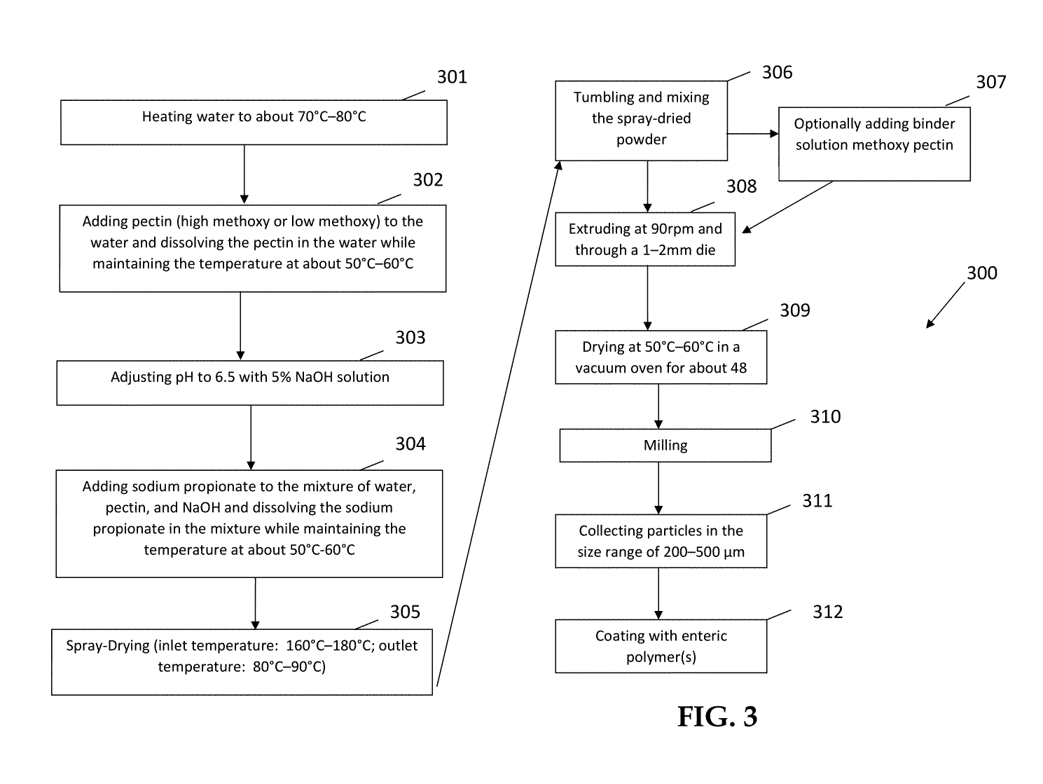

The process 300 for preparing enteric-coated microparticles is generally outlined in FIG. 3 and described in more detail below.

Entrapment: A 1500 g (1.5 kg) batch of low methoxyl pectin (5% aqueous pectin solution, obtained from CPKelco, Atlanta, Ga. was prepared. In particular, in step 301, 1425 grams of water were weighed and heated to about 70° C. to about 80° C. Subsequently in step 302, 75 grams of low methoxyl pectin was added to the water and dispersed in the water and allowed to dissolve while maintaining the temperature between about 50° C. and about 60° C. Then in step 303, the pH was adjusted to about 6.5 with 5% NaOH, after which 75 grams of sodium propionate was added and allowed to dissolve while maintaining the temperature of the solution between about 50° C. and about 60° C. in step 304. In step 305, the solution was spray-dried using a Buchii mini spray dryer model B290 at an inlet temperature from about 160° C. to about 180° C. and an outlet temperature from about 80° C. to about 90° C. This provided a spray-dried powder where the propionate was physically entrapped in a low methoxyl pectin matrix.

Extrusion: In step 306, 150 grams of the spray-dried powder were tumbled in a batch Hobart tumbler at a speed setting of 1 and paddle mixed in a Hobart bowl mixer while, in optional step 307, about 100 g of binder solution with calcium cross linker was sprayed on the spray-dried powder to crosslink the pectin matrix. The binder solution used was an aqueous 10% maltodextrin having a dextrose equivalent (DE) of 10 and 1% calcium chloride. In step 308, the resulting material was fed into an LCI extruder at 90 rpm and through a 1-2 mm die. In step 309, the extrudates were collected and dried at about 50° C. to about 60° C. in a vacuum oven for about 48 hours. When dried to a moisture content of less than about 5%, the dried extrudate was milled in a Waring blender in step 310 and then sifted in step 311 to collect particles having a mean particle size of about 200 to about 500 microns for further processing.

Enteric coating: In step 312, three coats were applied to the particles—(1) 15% solution of ethyl cellulose (inner coat), 10% zein solution (middle coat), and (3) 10% shellac solution (outer coat).

The following formulation was used to prepare the ethyl cellulose-containing layer of the enteric coating: ethyl alcohol (200 proof; 247.5 g); deionized water (27.5 g); ethyl cellulose 4 std (12.5 g); ethyl cellulose 10 std (12.5 g). (4 std and 10 std designate grades of ethyl cellulose, in particularly the ethoxyl type, obtained from the Dow Chemical Company, Midland, Mich.) The ethyl alcohol and deionized water were mixed, and then ethyl cellulose was added and mixed to form a solution. Then 211 grams of this solution was used to coat about 90 g of the spray-dried powder according to the coating procedure described in more detail below.

The following formulation was used to prepare the zein-containing layer of the enteric coating: ethyl alcohol (200 proof; 126 g), deionized water (54 g), and zein (20 g). In particular, the ethyl alcohol and deionized water were first mixed. Zein was added to the mixture of ethyl alcohol and deionized water and mixed to dissolve the zein to form a solution. Then 145 grams of this solution was used to coat about 130 g of the spray-dried powder.

The following formulation was used to prepare the shellac-containing layer of the enteric coating: 25% shellac aqueous solution (75 grams) from Temuss Products Ltd. (Canada) and deionized water (75 grams). Specifically, the 25% shellac aqueous solution was diluted to 12.5% with deionized water, and then 115 grams of this solution was used to coat 115 grams of the spray-dried powder.

The coating steps were carried out in a bench Mini Glatt fluid bed coater with a bottom spray Wurster process. The product temperature was about 30° C. to about 45° C. and the coating spray rate was from about 1 g/min to about 2 g/m. The coating parameters during the ethyl cellulose coating were similar to the parameters during the zein and shellac coating steps.

The compositions including propionate embedded in the pectin and encapsulated in the ethyl cellulose/zein/shellac coating were analyzed for percent propionic acid content using High Performance Liquid Chromatography (HPLC).

Sample preparation prior to injecting into the HPLC instrument included hydrating the samples with water adjusted to a pH of 7.5 (with 5% NaOH or KOH solution) and applying a high shear to facilitate degradation of the coating and disintegration of the pectin matrix. The treatment was applied for a time sufficient to release the propionate from the pectin matrix.

For the HPLC, a 300 mm long and 7.8 mm in diameter Bio Rad organic acid column (HPX-87H (acid form)) with a polystyrene-divinylbenzene sulfonic acid resin was used. The mobile phase was 3 mM nitric acid. The flow rate was 0.6 ml/min at 65° C. A refractive index detector was used.

The low methoxyl pectin samples were found to include 24.57% propionic acid by weight of the enteric-coated composition.

Dissolution test: A dissolution test was conducted to evaluate the release profile of the enteric-coated microparticles when incubated at stomach and intestinal pHs.

Sample 1: 1 gram of the enteric-coated microparticles was dispersed in 50 grams of deionized water, and the pH was adjusted to 3.0 by adding concentrated hydrochloric acid. The sample was then incubated at 37° C. for 45 minutes in a water bath with constant shaking to simulate passage of the composition through the human stomach. At the end of the incubation period, a sample was filtered with a 0.45 micron filter and analyzed by HPLC for propionic acid content.

Sample 2: This sample was treated according to the procedure of Sample 1 but then after incubation at pH 3.0, the pH of Sample 2 was neutralized to about pH 7.0 by addition of sodium bicarbonate solution. Sample 2 was incubated in a shaker at 37° C. for 6 hours at pH 7.0 to simulate passage of the composition through the small intestine. The sample was then filtered and analyzed by HPLC for propionic acid content.

Sample 3: This sample was treated according to the procedure of Sample 1, followed by the procedure of Sample 2, and then incubated for an additional 24 hours at pH 7.0 to simulate passage of the composition through the large intestine. The sample was then filtered and analyzed by HPLC for propionic acid content. The results were as follows:

TABLE 1

| | | Sample 3 |
|---|---|---|
| | Sample 2 | (45 minutes at pH 3.0; |
| Sample 1 | (45 minutes at pH 3.0; | 6 hours at pH 7.0; |
| (45 minutes at pH 3.0) | 6 hours at pH 7.0) | 24 hours at pH 7.0) |
| 0.06% propionic acid | 8.25% propionic acid | 18.8% propionic acid |

As shown in the table above, negligible release of propionate was observed in the simulated stomach, some release of propionate was observed in the simulated small intestine, and significantly larger release of propionate was observed in the simulated large intestine. The percentages in Table 1 represent percentage of propionic acid by total weight of the enteric-coated microparticles.

### Example 2

### Use of High Methoxyl Pectin to Entrap Propionate for Targeted Delivery to the Large Intestine

The process for preparing enteric-coated microparticles is generally outlined in FIG. 3 and described in more detail below.

Entrapment: A 2 kg batch of high methoxyl pectin (7% pectin solution) was prepared. Water (1860 grams) was heated to a temperature of about 70° C. to about 80° C., and high methoxyl pectin, obtained from Cargill, Inc., Minneapolis, Minn. (140 grams) was dispersed in the water and allowed to dissolve while maintaining a temperature at about 50° C. to about 60° C. The pH was adjusted to 6.5 with 5% NaOH solution, after which 140 grams of sodium propionate were added and allowed to dissolve while maintaining the temperature of the solution at about 50° C. to about 60° C. The resulting solution was spray-dried using a Buchii mini spray dryer model B290 at an inlet temperature of about 160° C. to about 180° C. and an outlet temperature of about 80° C. to about 90° C. This resulted in a spray-dried powder where the propionate is embedded in the high methoxyl pectin matrix.

Extrusion: The extrusion was carried out as described above in Example 1, except that the binder solution of 10% maltodextrin having a dextrose equivalent (DE) of 10 did not contain calcium chloride for cross-linking the pectin.

Enteric coating: The enteric coating was carried out as described above in Example 1.

The coated samples from Example 2 were analyzed by HPLC for percent propionic acid content as described in Example 1. Sample preparation prior to injecting into the HPLC instrument included hydrating samples with water adjusted to a pH of 7.5 (e.g., with 5% NaOH or KOH solution) and applying a high shear to facilitate degradation of the coating and disintegration of the pectin matrix. The treatment was applied for a time sufficient to release the propionate from the high methoxyl pectin matrix. The high methoxyl pectin samples were observed to include about 20.41% propionic acid by total weight of the enteric-coated composition.

Dissolution test: The dissolution test was carried out as described in Example 1. The results are presented in Table 2:

TABLE 2

| | | Sample 3 |
|---|---|---|
| | Sample 2 | (45 minutes at pH 3.0; |
| Sample 1 | (45 minutes at pH 3.0; | 6 hours at pH 7.0; |
| (45 minutes at pH 3.0) | 6 hours at pH 7.0) | 24 hours at pH 7.0) |
| 0.05% propionic acid | 22.8% propionic acid | 20.2% propionic acid |

As shown in Table 2 above, negligible release of propionate was observed in the simulated stomach and significantly more release of propionate was observed in the simulated small intestine. A slightly lower release of propionate was observed in the simulated large intestine as compared to the simulated small intestine but significantly more release as compared to the amount released in the simulated stomach.

### Example 3

### In Vitro Digestion Evaluation

The release of short chain fatty acids in the human gastrointestinal tract from enteric-coated compositions and the effect of the compositions on the gut microbiota was investigated.

The passage through the different areas of the gastrointestinal tract was simulated through use of the Simulator of Human Intestinal Microbial Ecosystem (“SHIME®”) technology platform. A sample SHIME setup is discussed in more detail, for example, in K. Molly et al., “Development of a 5-step multichamber reactor as a simulation of the human intestinal microbial ecosystem,” Applied Microbiology and Biotechnology, 39(2): 254-258 (1993), incorporated by reference herein in its entirety. The SHIME setup was designed to provide an in vitro system to analyze the microbial community in the colon, including which microbes are present and in what quantities and the by-products they produce. This approach is relatively fast and a much less expensive approach than testing in animals and humans.

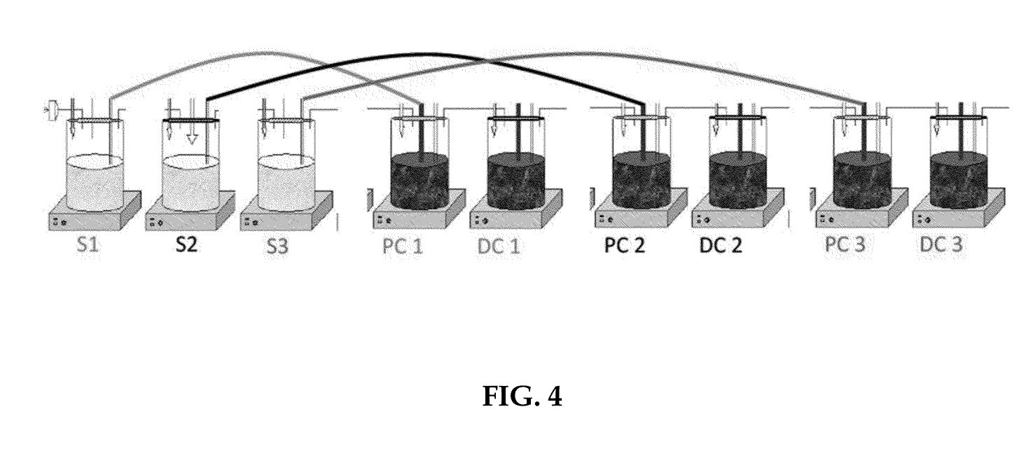

FIG. 4 illustrates an exemplary modified SHIME® setup including reactors set up to mimic the temperature and pH of the human digestive tract. The setup was used to evaluate the concentration of propionate, acetate, butyrate, ammonium, and lactate; intestinal pH variation; total bacteria; and quantities of Bifidobacteria, Lactobacilli, Firmicutes, and Bacteroidetes in three different locations: (1) stomach+small intestine (“S”); (2) proximal colon (“PC”); and (3) distal colon (“DC”). This evaluation complements the bench chemistry assessment that was done to demonstrate the controlled delivery of the propionic acid and pectin to the colon. Use of the SHIME setup demonstrated that both propionate and the pectin are being delivered to the PC and DC and modulated the microbial community and its by-products in a positive fashion as based on present understanding.

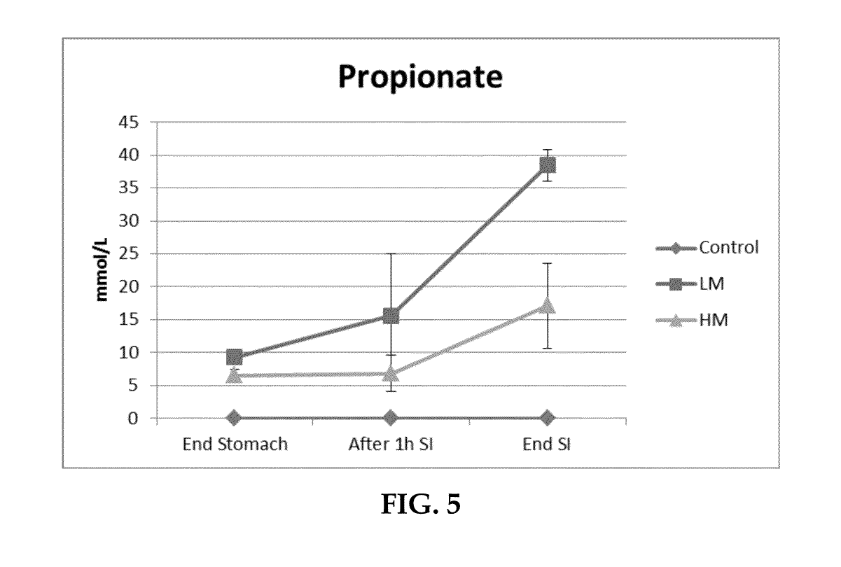

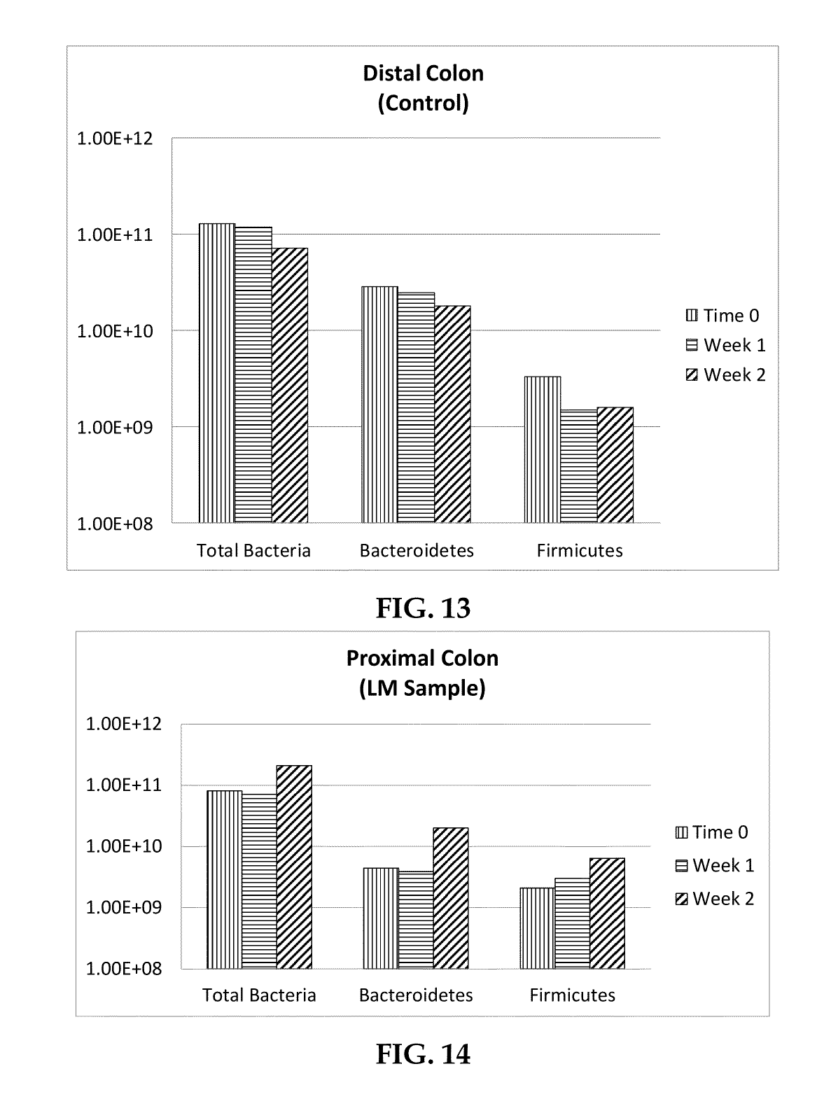

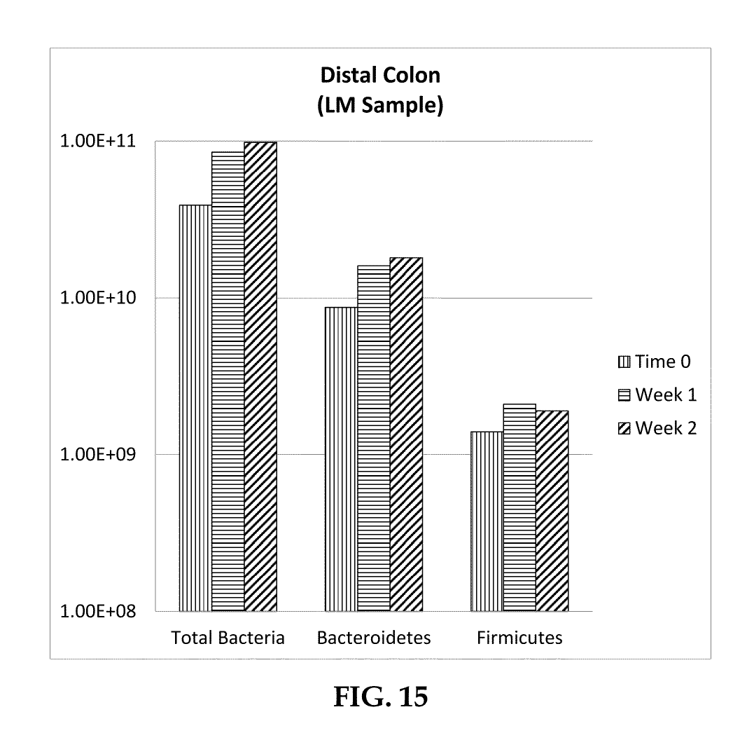

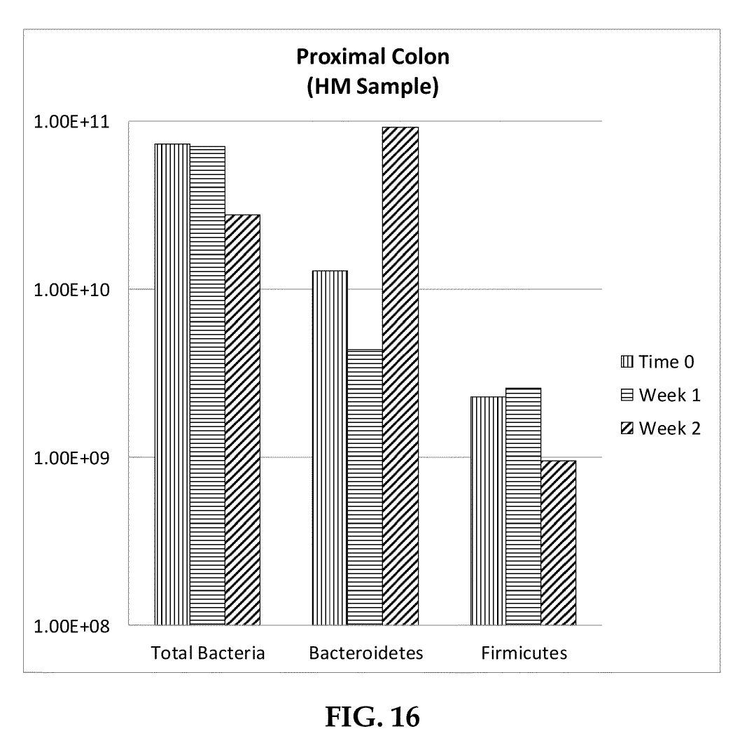

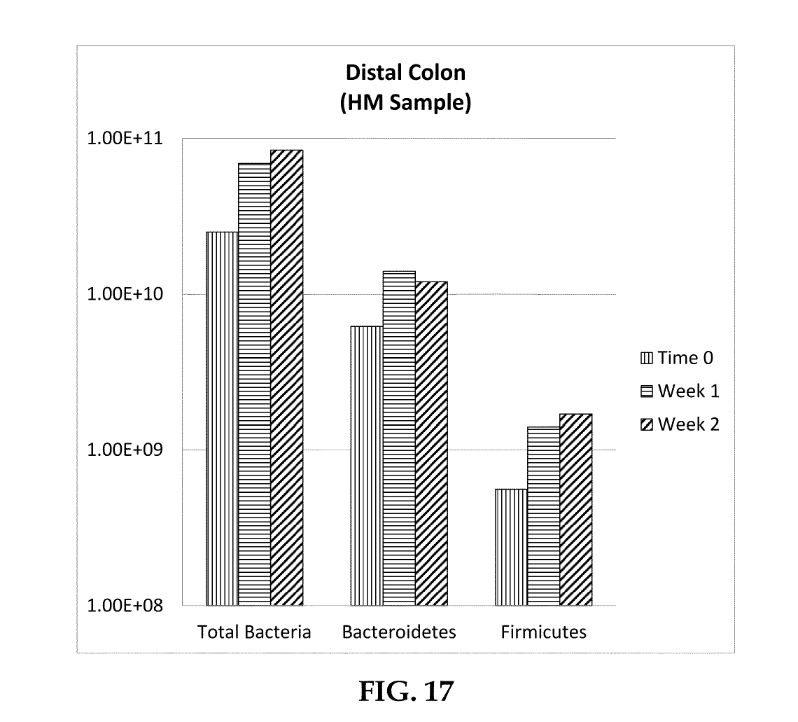

As can be seen in FIG. 4, the exemplary modified SHIME® setup uses one reactor “S” for the stomach and the small intestine, one reactor “PC” for the proximal colon), and one reactor “DC” for the distal colon. The simulated digestive tracts were set up in triplicate and run simultaneously. This SHIME setup was used to generate the results shown in FIGS. 5-19. The in vitro digestion evaluation setup was as follows.

All reactors were held at 37° C. to mimic the human body. The “S” reactors simulate the stomach in temperature, pH, and includes pancreatic enzyme/bile solution. The “PC” reactors simulate the proximal colon in temperature, human microbiota, pH, anaerobic conditions, and turnover rate. The “DC” reactors simulate the distal colon in temperature, human microbiota, pH, anaerobic conditions, and turnover rate.

Each “S” reactor was linked to a “PC” reactor, which was linked to a “DC” reactor to represent the human digestive process (S1-PC1-DC1; S2-PC2-DC2; and S3-PC3-DC3). The PC and DC reactors were maintained anaerobically by flushing the headspace with N₂ and continuously stirred. The reactors in each series were connected via tubing connected to a peristaltic pump. The rate at which the contents of the reactors flowed from start to finish was intended to mimic human digestion through the use of the peristaltic pumps connecting the reactors. The pHs in the reactors were also adjusted to match each segment of the digestive tract. The “S” reactors had an initial pH of about 2.0 and a final pH of about 7.5. The PC reactor had a pH between about 5.6 to about 5.9. The “DC” reactors had a pH of about 6.6 to about 6.9. The pH was controlled in the “DC” and “PC” reactors by the addition of appropriate quantities of acid or base.

Start-up (3 weeks): The nine reactors were inoculated with a fecal sample taken from a healthy male, 30 years old with no history of antibiotics in the last six months. Each reactor was run for a three week start-up period, which allowed the microbial community to differentiate and stabilize in the reactors prior to the beginning of the experimental treatment.

Treatment period (2 weeks): To start the digestion process after the three week start-up period, standard SHIME feed (starting with 140 mL) was dosed to the three “S” reactors three times per day to simulate breakfast, lunch and dinner. The “S1,” “PC1,” and “DC1” reactors (FIG. 4) acted as controls to determine the baseline microbial community composition and activity. During the breakfast feed, the “S2” and “S3” reactors also received a low methoxyl pectin (“LM”) sample made according to Example 1 and high methoxyl pectin (“HM”) sample made according to Example 2, respectively, to determine the effect of the enteric-coated microparticles on the microbial community composition and activity. The enteric-coated microparticles were dosed at 2 grams per day during the breakfast feeding only. No enteric-coated microparticles were administered at lunch or dinner. The control “S” reactor received no enteric-coated microparticles at any feeding.

In each of the “S” reactors, the feed included arabinogalactan (1.2 g/L), pectin (2 g/L), xylan (0.5 g/L), glucose (0.4 g/L), yeast extract (3 g/L), peptone (1 g/L), mucin (3 g/L), L-cysteine-HCl (0.5 g/L), and starch (4 g/L) in water. The SHIME feed was added and held in the “S” reactors at pH 2.0 for 1.5 hours, after which 60 mLs of a pancreatic enzyme and bile salts solution was added (6 g/L Oxgall (Difco, Bierbeek, Belgium), 1.9 g/L pancreatin (Sigma, Bornem, Belgium), and 12.5 g/L NaHCO₃). This brought the pH of the “S” reactors to 7.5 and the material in the reactors was held for an additional 2.5 hours before beginning to pump the contents of each “S” reactor into the corresponding “PC” reactor.

The volume in each “PC” reactor was held constant at 500 mL with the addition of the contents of the corresponding “S” reactor. This pumping from the “S” reactors was completed in 20 hours (turnover time), and then the contents were pumped from each “PC” reactor to the corresponding DC reactor. The volume in the PC reactors was held constant at 500 mL with the addition of the contents of the respective “S” reactors with a turnover time of 20 hours. The volume in the DC reactors was held constant at 800 mL with a turnover time of 36 hours. Contents of the DC reactors were removed as needed by pump to maintain the constant volume.

Liquid samples (10 mL) from each colon reactor were collected and frozen at −20° C. for subsequent analysis. The SCFA were extracted from the samples with diethyl ether and determined with a Di200 gas chromatograph (GC; Shimadzu's-Hertogenbosch, The Netherlands). The GC was equipped with a capillary free fatty acid packed column (EC-1000 Econo-Cap column (Alltech, Laarne, Belgium), 25 m×0.53 mm; film thickness 1.2 microns), a flame ionisation detector and a Delsi Nermag 31 integrator (Thermo Separation Products, Wilrijk, Belgium). Nitrogen was used as the carrier gas at a flow rate of 20 ml/min. The column temperature was set at 130° C. and the temperature of the injector and detector was set at 195° C. The frozen liquid samples from each colon reactor were also analyzed for ammonium using a 1026 Kjeltec Auto Distillation (FOSS Benelux, Amersfoort, The Netherlands). Ammonium in the sample was liberated as ammonia by the addition of an alkali (MgO). The released ammonia was distilled from the sample into a boric acid solution. The solution was backtitrated using a 665 Dosimat (Metrohm, Berchem, Belgium) and 686 Titroprocessor (Metrohm).

The bacterial concentrations in the reactors were measured by quantitative PCR using species specific primers to amplify 16S rRNA genes.

As can be seen in FIG. 5, minimal release of propionate occurs in the simulated stomach and release increases towards the end of the simulated small intestine. Less propionate was released in the high methoxyl pectin sample than the low methoxyl pectin sample by the end of the simulated small intestine.

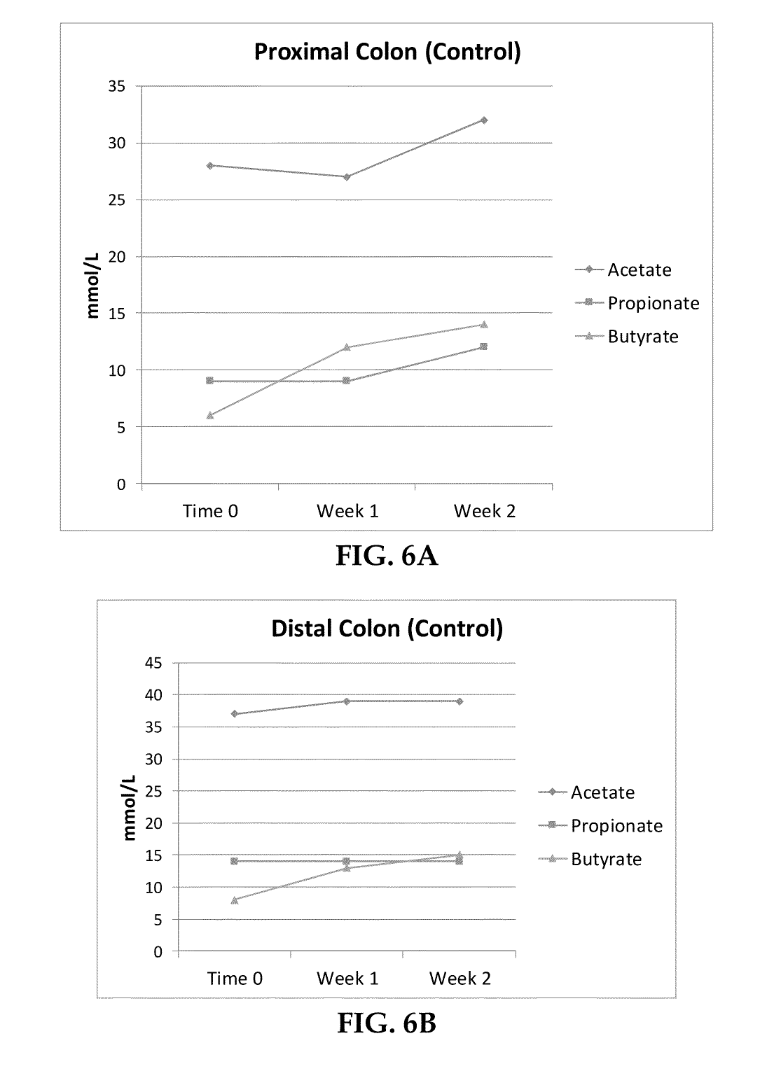

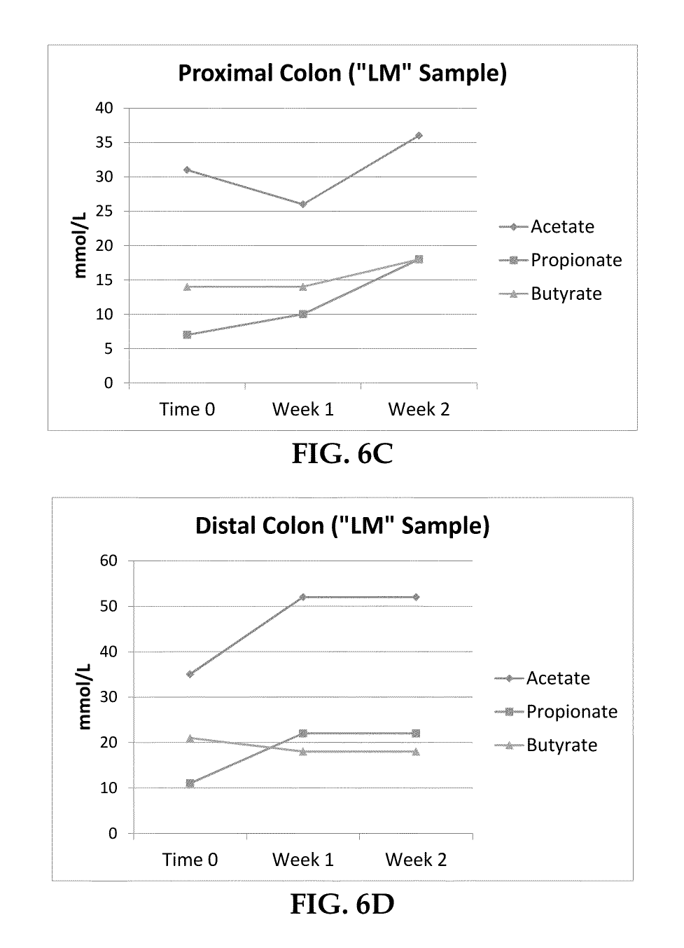

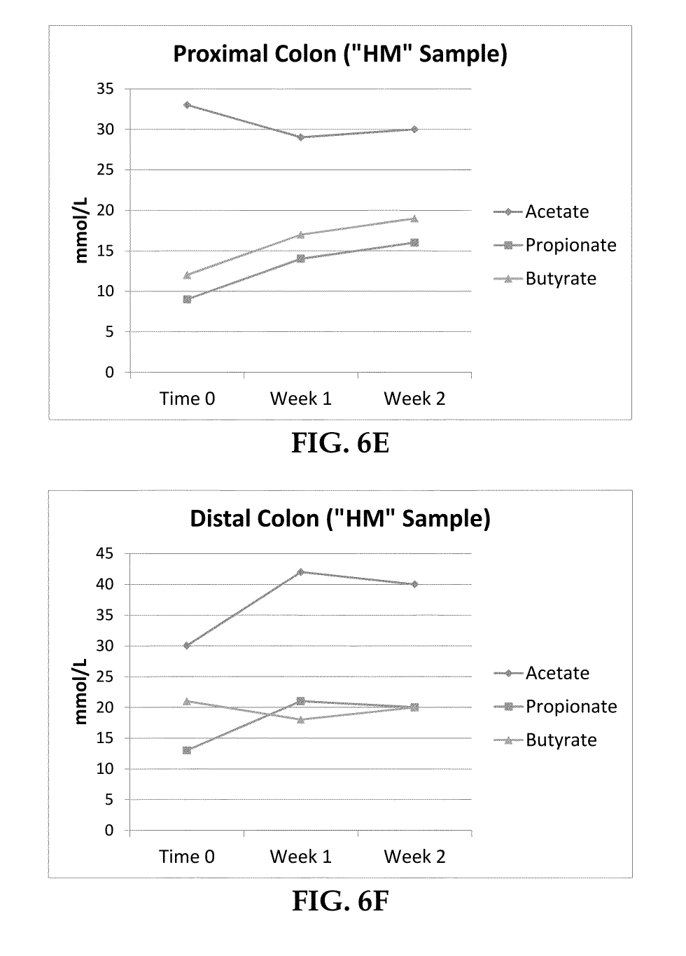

As can be seen in FIGS. 6A-6F, both the low methoxyl pectin and high methoxyl pectin samples improved the concentration of propionate as compared to the control samples, especially in the distal colon in the simulated proximal colon (PC) and the simulated distal colon (DC). Lactate is normally a transient metabolite which acts as an intermediate of production for propionate and butyrate (e.g., in metabolic cross-feeding, some bacteria may utilize a substrate like pectin to produce lactate, while other bacteria may utilize the lactate to produce butyrate or other short chain fatty acids).

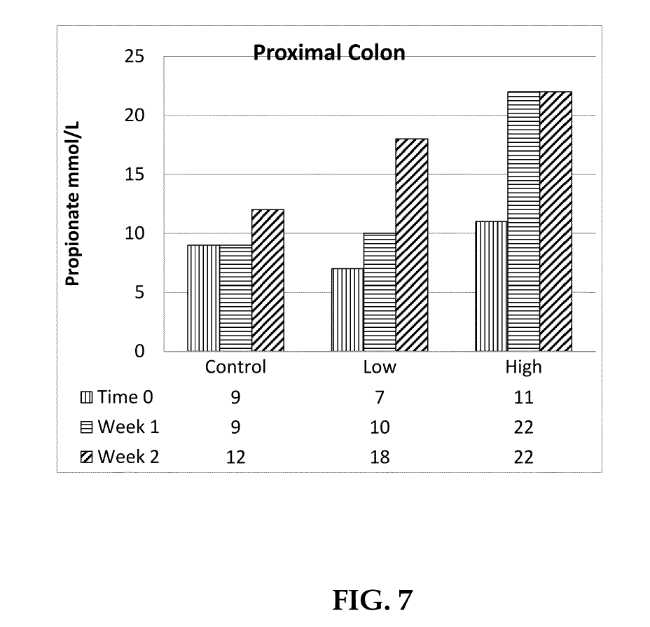

As can be seen in FIGS. 7 and 8, a comparison of propionate concentration in the simulated proximal colon and distal colon shows that the LM microparticles and the HM samples led to a higher concentration of propionate as compared to the control over a two week time period.

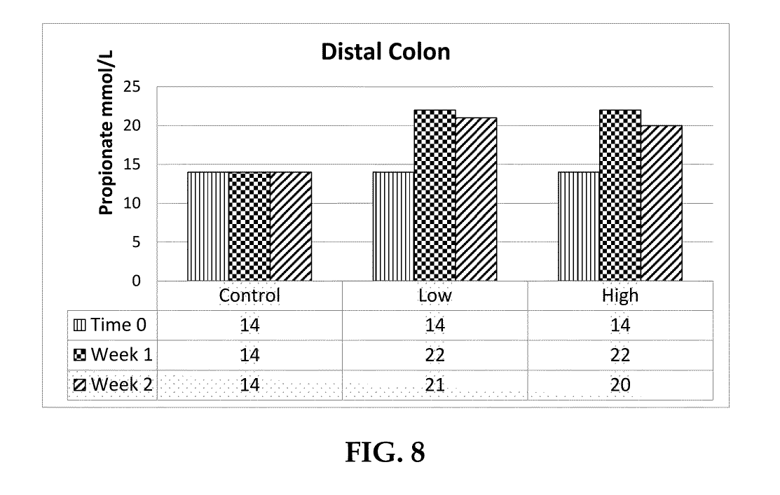

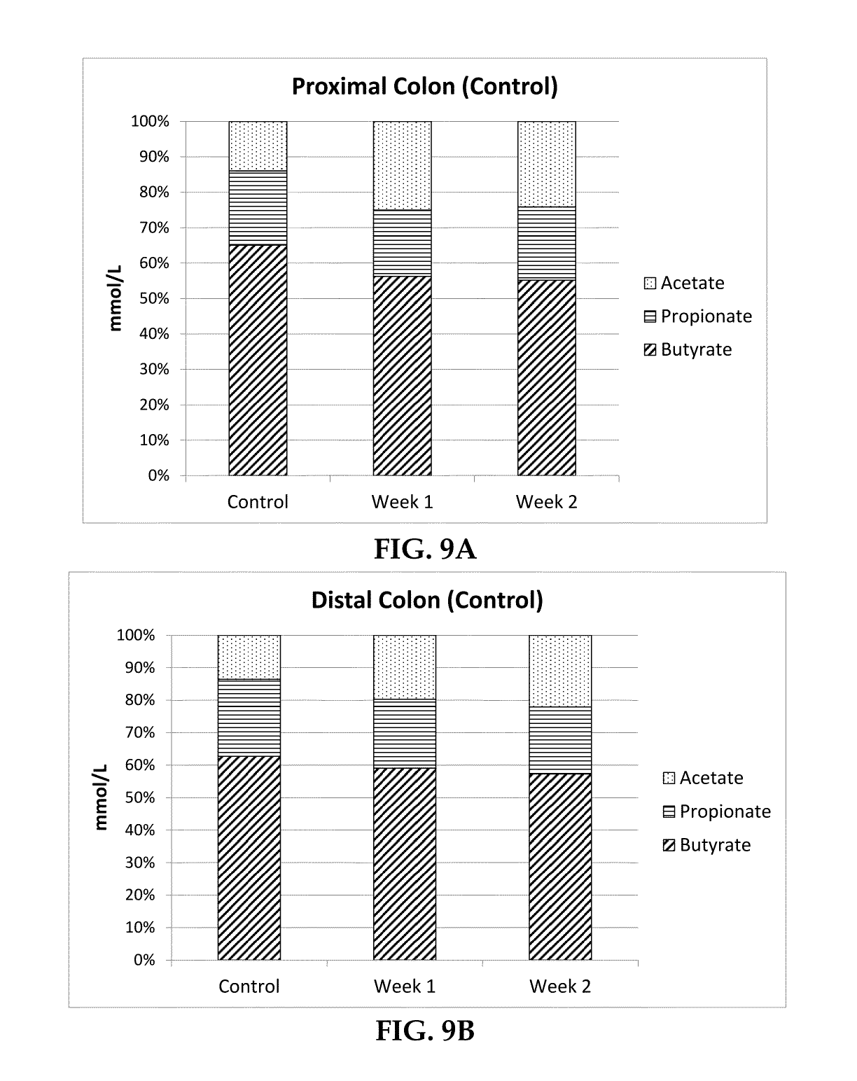

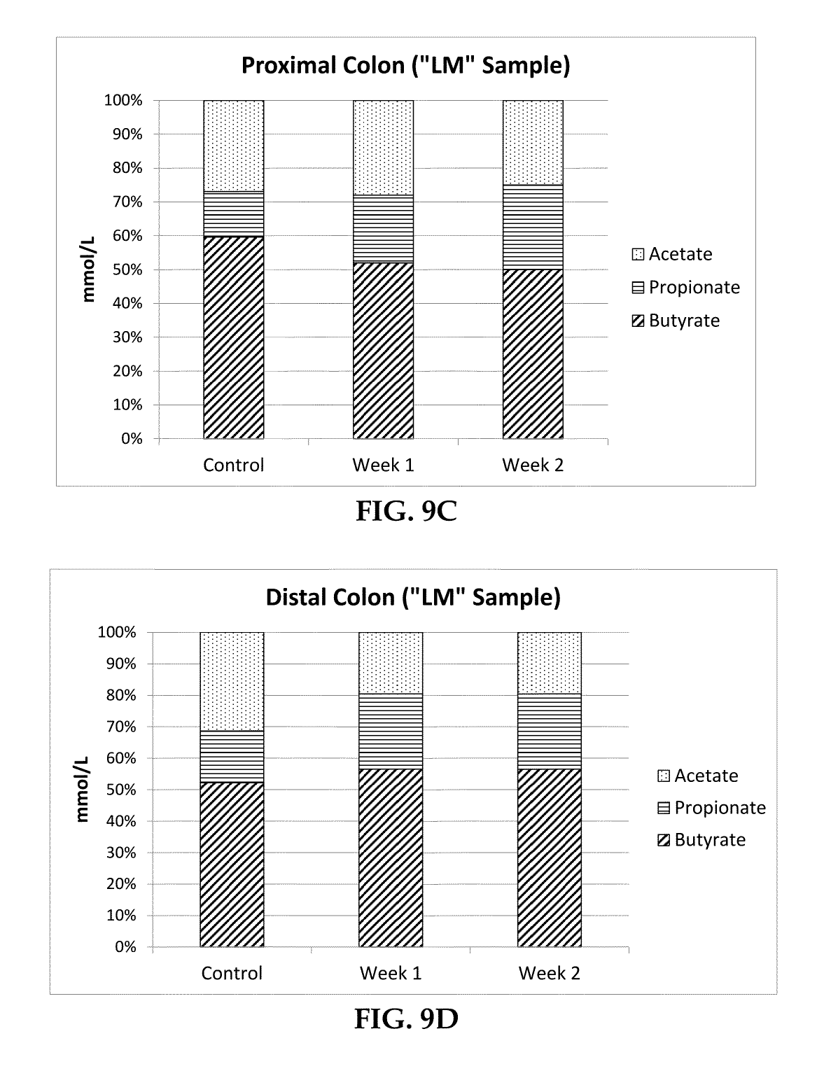

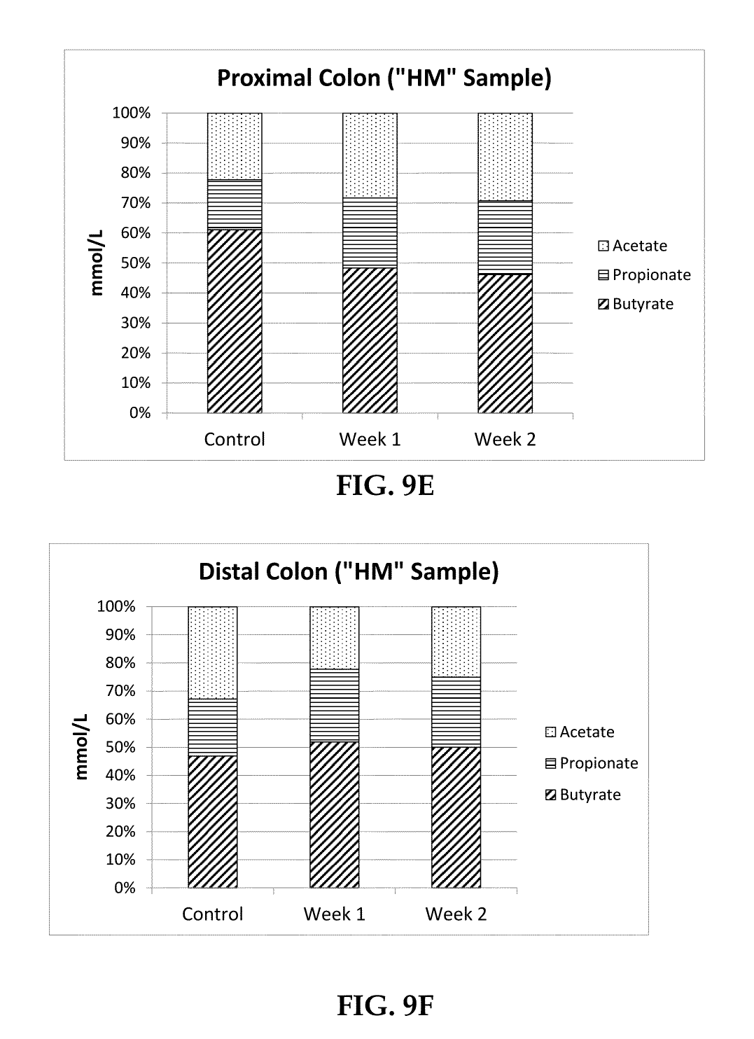

In FIGS. 9A-9F, the percentages of acetate, propionate, and butyrate over two weeks are measured in the simulated proximal and distal colons for the control, HM samples, and LM samples. The results show that treatment with the LM and HM samples resulted in increased propionate in both the simulated proximal colon and simulated distal colon, which indicates that the pectin of the microparticles is being degraded by intestinal bacteria. Generally, the presence of acetate, butyrate and propionate suggest that the gut microbial community is healthy and converting lactate into the three main SCFA's: acetate, butyrate, and propionate.

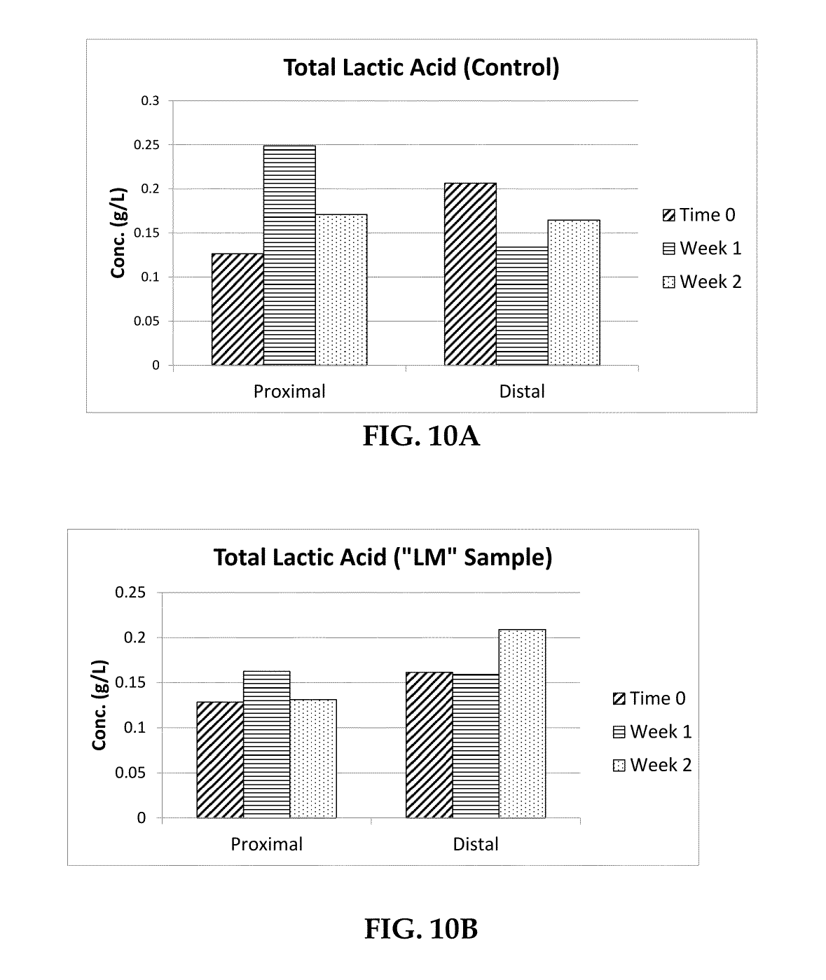

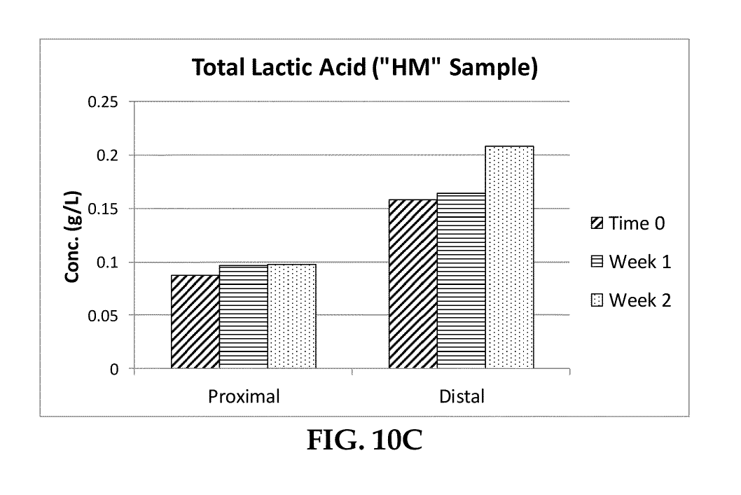

FIGS. 10A-10C show the concentrations of total lactic acid in the simulated proximal colon and the distal colon at time zero, week 1, and week 2. This is an indication that the pectin and propionate are becoming available in the distal colon after two weeks to modulate the microbial community (e.g., an increase in the number of microbes) to produce more lactate. The lactate is an intermediate to SCFA production and also will create a lower pH environment. Higher pH in the colon is associated with an increase in the risk of colon cancer, so it is presently believed that the lowering of the pH is an a beneficial result.

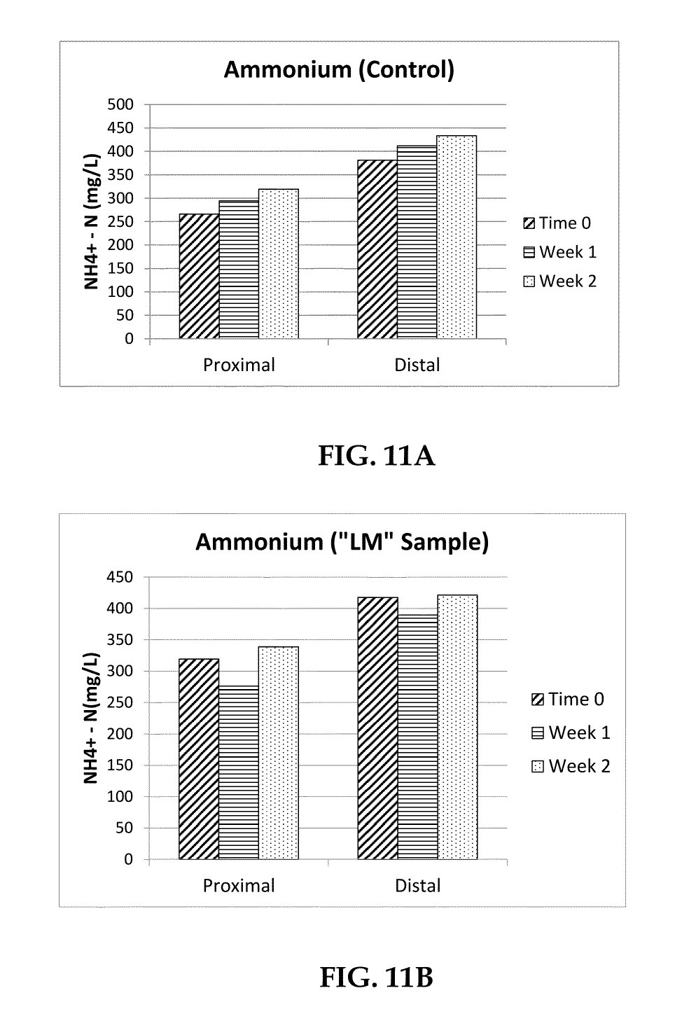

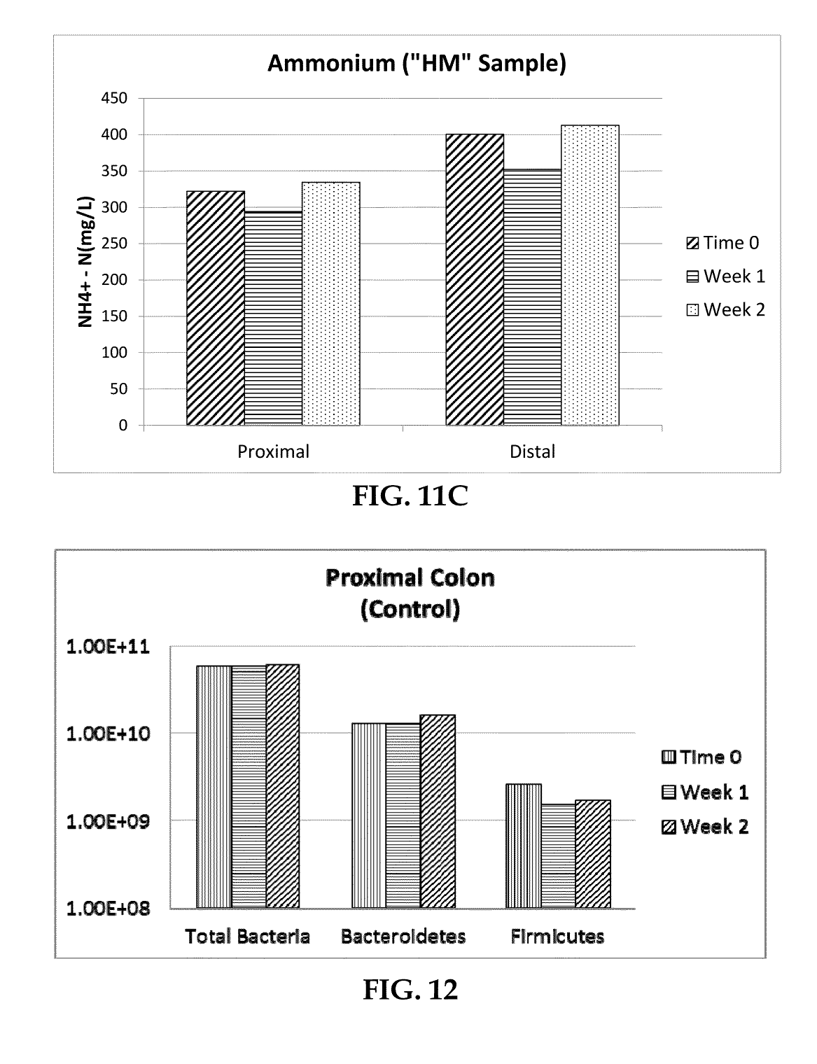

FIGS. 11A-11A show the ammonium concentrations in the simulated proximal and distal colon, with FIG. 11A showing results for the control, FIG. 11B showing results for the LM sample, and FIG. 11C showing results for the HM sample. Ammonium is a marker for proteolysis and may indicate the activity of bacteria in breaking down the pectin and/or short chain fatty acids. In the control, an increasing trend was observed along the 2 weeks of experiment, while in presence of the treatment with LM and HM, a decrease in ammonium concentration was observed at one week, and then an increase at two weeks which brought the total ammonium concentration to similar levels as the start of the experiment.

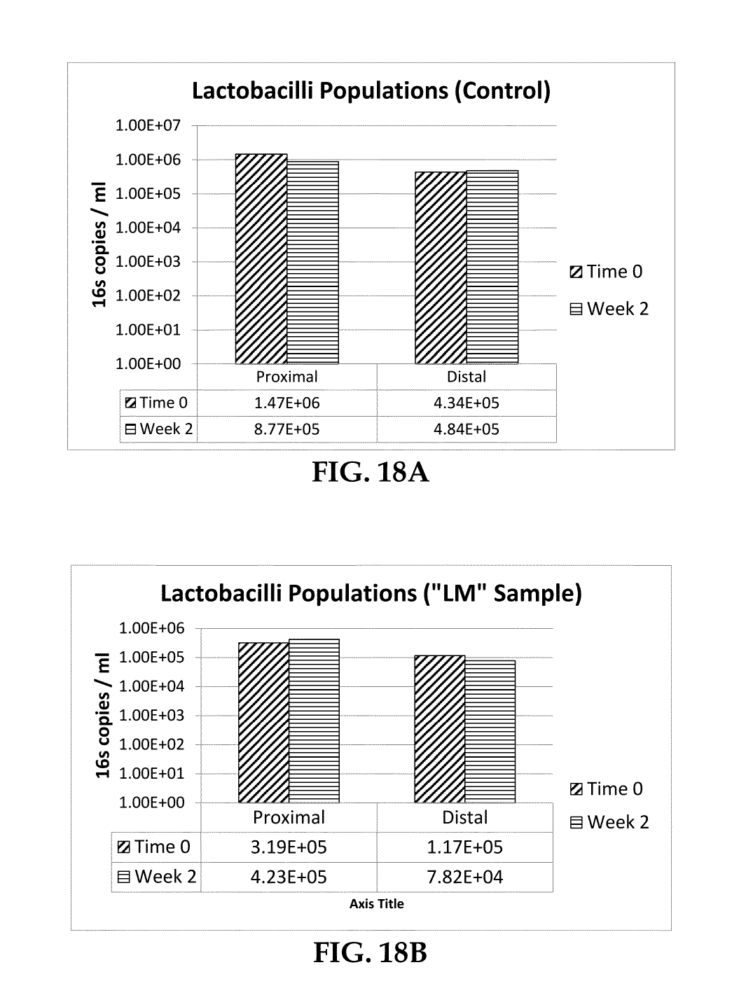

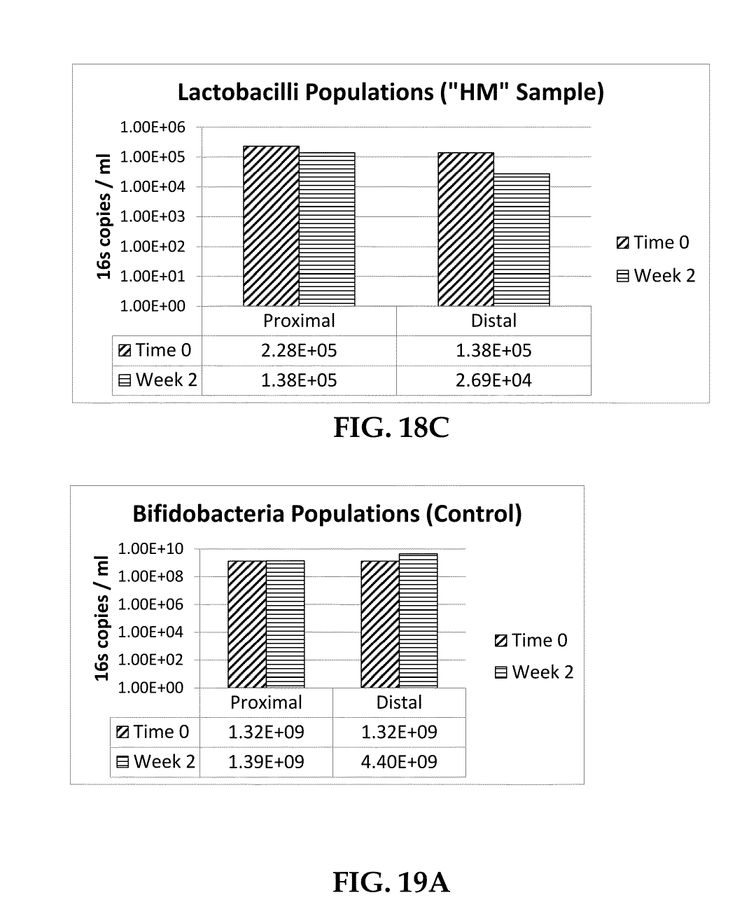

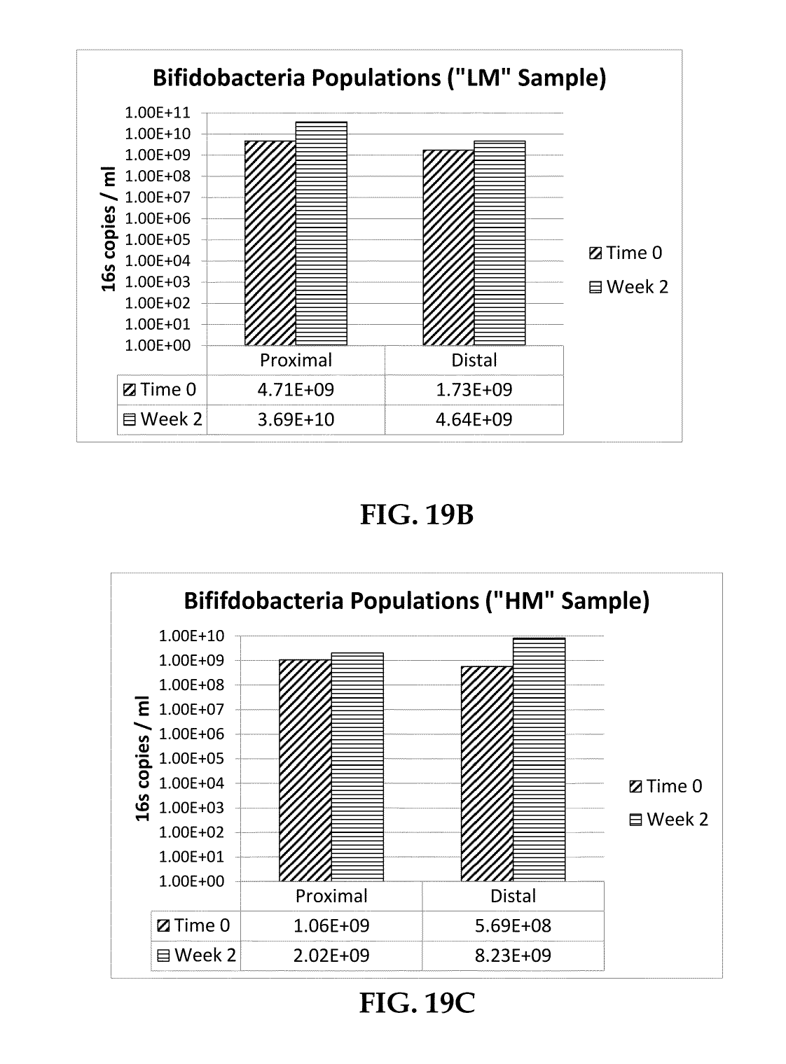

FIGS. 12-17 show the concentration of total bacteria (as measured by copies of the 16s rRNA gene is amplified and quantified by qPCR), Bacteroidetes bacteria and Firmicutes bacteria in the proximal and distal colon before treatment (time 0) and after one and two weeks of treatment with a control sample, low methoxyl pectin sample, or high methoxyl pectin sample. LM and HM treatments led to an increase in the concentration of total bacteria in the simulated distal colon, whereas the control treatment led to decreased total bacteria. The results also show that the increase in total bacteria in the simulated distal colon during the treatment period was mainly correlated with an increase in Bacteroidetes bacteria. LM and HM treatments also led also to a slight increase in Firmicutes bacteria, whereas a statistically significant decrease was observed for the control treatment. FIGS. 18A-18C show a general trend of the dynamic state of Lactobacilli populations. FIGS. 19A-19C show a slight increase in the concentration of bifidobacteria over time in the simulated proximal and distal colons for the control sample. However, for the LM and HM samples, the increases were greater.

The foregoing descriptions are not intended to represent the only forms of the enteric-coated compositions. Similarly, while methods have been described herein in conjunction with specific embodiments, many alternatives, modifications, and variations will be apparent to those skilled in the art in light of the foregoing description.

What is claimed is:

1. A functional food ingredient comprising:
a fermentation precursor matrix comprising a metabolite entrapped in the fermentation precursor matrix; and
an enteric coating that encapsulates the fermentation precursor matrix with the entrapped metabolite.

2. The composition of claim 1, wherein the fermentation precursor matrix comprises at least one polysaccharide selected from the group consisting of pectin, alginate, xylan, guar gum, or a combination thereof.

3. The composition of claim 1, wherein the metabolite is selected from the group consisting of propionic acid or salt thereof, butyric acid or salt thereof, acetic acid or salt thereof, lactic acid or salt thereof, succinic acid or salt thereof, or a combination thereof.

4. The composition of claim 1, wherein the metabolite comprises sodium propionate and the fermentation precursor matrix comprises high methoxyl pectin.

5. The composition of claim 4, wherein the high methoxyl pectin matrix is crosslinked with a metal divalent or trivalent cation.

6. The composition of claim 1, wherein the metabolite comprises sodium propionate and the fermentation precursor matrix comprises pectin.

7. The composition of claim 1, wherein the enteric coating comprises one or more enteric coating materials selected from the group consisting of shellac, zein, and ethyl cellulose.

8. The composition of claim 1, wherein the enteric coating includes an inner layer comprising ethyl cellulose, a middle layer comprising zein, and an outer layer comprising shellac.

9. A food product comprising an effective amount of the functional food ingredient of claim 1.

10. The food product of claim 9, wherein the food product is a chewing gum, biscuit, cookie, powder beverage, chocolate, or confection.

11. The food product of claim 10, wherein the fermentation precursor matrix comprises at least one polysaccharide selected from the group consisting of pectin, alginate, xylan, guar gum, or a combination thereof, and the metabolite is selected from the group consisting of propionic acid or salt thereof, butyric acid or salt thereof, acetic acid or salt thereof, lactic acid or salt thereof, succinic acid or salt thereof, or a combination thereof.

12. A pharmaceutical or nutraceutical composition comprising an effective amount of the functional food ingredient of claim 1.

13. The pharmaceutical or nutraceutical composition of claim 12, wherein the fermentation precursor matrix comprises at least one polysaccharide selected from the group consisting of pectin, alginate, xylan, guar gum, or a combination thereof, and the metabolite is selected from the group consisting of propionic acid or salt thereof, butyric acid or salt thereof, acetic acid or salt thereof, lactic acid or salt thereof, succinic acid or salt thereof, or a combination thereof.

14. A method of suppressing appetite of a human subject, the method comprising administering to the human subject an enteric-coated composition comprising:
a fermentation precursor matrix comprising a metabolite entrapped in the fermentation precursor matrix; and
an enteric coating that encapsulates the fermentation precursor matrix with the entrapped metabolite.

15. The method of claim 14, wherein the fermentation precursor matrix comprises at least one polysaccharide selected from the group consisting of pectin, alginate, xylan, guar gum, or a combination thereof, and the metabolite is selected from the group consisting of propionic acid or salt thereof, butyric acid or salt thereof, acetic acid or salt thereof, lactic acid or salt thereof, succinic acid or salt thereof, or a combination thereof.

16. The method of claim 14, wherein the metabolite comprises sodium propionate and the fermentation precursor matrix comprises pectin.

17. The method of claim 14, wherein the enteric coating comprises one or more enteric coating materials selected from the group consisting of shellac, zein, and ethyl cellulose.

18. The method of claim 14, wherein the enteric coating includes an inner coat formed of ethyl cellulose, a middle coat formed of zein, and an outer coat made of shellac.

19. A method of making a functional food ingredient, the method comprising:
heating an aqueous liquid to a temperature of about 50° C. to about 80° C.;
adding a fermentation precursor to the heated aqueous liquid to form a first mixture;
adding a metabolite to the first mixture to form a second mixture;
drying the second mixture to form a powder;
milling the dried powder to provide particles; and
coating the particles with an enteric coating.

20. The method of claim 19, wherein the fermentation precursor matrix comprises at least one polysaccharide selected from the group consisting of pectin, alginate, xylan, guar gum, or a combination thereof.

21. The method of claim 19, wherein the metabolite is selected from the group consisting of propionic acid or salt thereof, butyric acid or salt thereof, acetic acid or salt thereof, lactic acid or salt thereof, succinic acid or salt thereof, or a combination thereof.

22. The method of claim 20, wherein the polysaccharide comprises low methoxyl or high methoxyl pectin.

23. The method of claim 19, further comprising adding a binder solution including a metal divalent or trivalent cation after the drying step.

24. The method of claim 19, further comprising adjusting a pH of the first mixture to about 6.0 to about 7.5 prior to adding the metabolite to the first mixture.

25. The method of claim 19, wherein the coating of the particles comprises coating the particles with an enteric coating including at least one of shellac, zein, and ethyl cellulose.

26. The method of claim 19, wherein the coating of the particles includes coating the particles with an enteric coating comprising an inner layer comprising ethyl cellulose, a middle layer comprising zein, and an outer layer comprising shellac.

27. The method of claim 19, wherein the particles have a mean particle size of between about 200 to about 500 microns.
